(19) United States
(12) Patent Application Publication
(10) Pub. No.: US 2024/0068042 A1
(43) Pub. Date: Feb. 29, 2024

(54) PROGNOSTIC METHODS FOR PLATINUM-BASED CHEMOTHERAPEUTICS

(71) Applicant: The Jackson Laboratory, Bar Harbor, ME (US)

(72) Inventors: Edison T. Liu, Bar Harbor, ME (US); Francesca Menghi, Bar Harbor, ME (US)

(73) Assignee: The Jackson Laboratory, Bar Harbor, ME (US)

(21) Appl. No.: 18/272,260

(22) PCT Filed: Jan. 10, 2022
(86) PCT No.: PCT/US2022/011767

§ 371 Date: Jul. 13, 2023

Related U.S. Application Data
(60) Provisional application No. 63/232,907, filed on Aug. 13, 2021.
(60) Provisional application No. 63/137,853, filed on Jan. 15, 2021.

(51) Int. Cl. C12Q 1/6886; C12Q 1/6869

(52) U.S. Cl. CPC C12Q 1/6886; C12Q 1/6869; C12Q 2600/106; C12Q 2600/154; C12Q 2600/158

(57) ABSTRACT

Provided herein, in some embodiments, are novel methods for predicting response to a platinum-based chemotherapeutic agent in triple negative breast cancer and ovarian carcinoma.

### CROSS REFERENCE TO RELATED APPLICATIONS

This application is a national stage filing under 35 U.S.C. § 371 of International Patent Application Serial No. PCT/US2022/011767, filed Jan. 10, 2022, entitled “Prognostic Methods for Platinum-Based Chemotherapeutics,” which claims the benefit under 35 U.S.C. § 1 19(e) of U.S. Provisional Application No. 63/137,853, filed Jan. 15, 2021, entitled “Prognostic Methods for Platinum-Based Chemotherapeutics,” and U.S. Provisional Application No. 63/232,907, filed Aug. 13, 2021, entitled “Prognostic Methods for Platinum-Based Chemotherapeutics,” the contents of each of which are hereby incorporated by reference in their entirety.

### GOVERNMENT LICENSE RIGHTS

This invention was made with government support under P30CA034196 awarded by National Institutes of Health and W81XWH-17-1-0005 awarded by Department of Defense. The government has certain rights in the invention.

### BACKGROUND

BRCA1 (BReast CAncer gene 1) and BRCA2 (BReast CAncer gene 2) encode proteins that help repair damaged DNA. Variants of these BRCA1/BRCA2 genes have been associated with increased risk of several cancers, including breast cancer and ovarian cancer. About 55%-72% of women who inherit a harmful BRCA1 variant and about 45%-69% of women who inherit a harmful BRCA2 variant will develop breast cancer by 70-80 years of age; and about 39%-44% of women who inherit a harmful BRCA1 variant and about 11%-17% of women who inherit a harmful BRCA2 variant will develop ovarian cancer by 70-80 years of age.

Following surgery to remove a primary cancer, platinum-based drugs such as cisplatin, carboplatin and oxaliplatin are often prescribed as a first-line chemotherapy. While they are effective in some patients, their use is limited by their severe, dose-limiting side effects.

### SUMMARY

The present disclosure provides, in some embodiments, methods to guide the selection of cancer (e.g., breast or ovarian cancer) patients who will likely be ‘good responders’ or ‘poor responders’ to treatment with a platinum-based chemotherapy agent. Such methods may limit the number of ‘poor responders’ who are unnecessarily exposed to the harmful side-effects of platinum-based agents. The experimental data described herein demonstrate that a BRCA1 and BRCA2 mutational status accompanied by a certain immune signature can be a predictor of how a particular population of patients will respond to platinum-based chemotherapy.

As described in the Examples below, the present disclosure provides a ‘decision tree’ for predicting patient response to platinum-based chemotherapy. By considering BRCA1/2 pathogenic mutational status and the expression of an immune signature in BRCA1/2 wild-type tumors, the methods provided herein can be used to more accurately predict chemo-responsiveness than by using BRCA1/2 mutational status alone or homologous recombination deficiency (HRD) scores. For example, a response state was developed based on the presence of BRCA1/2 pathogenic mutations in a tumor and, in a subset of patients having a BRCA1/2 wild-type tumor, on the strength of a M1 macrophage immune signature. Based on this decision tree, each tumor (and thus each patient) was given an assessment of ‘good’ or ‘poor’ (FIG. 6A). Patients with tumors having mutations for either BRCA1 or BRCA2 are predicted to be ‘good responders’ to a platinum-based agent. Patients with BRCA1/2 wild type tumors are predicted to be ‘good responders’ only when the M1 macrophage signal is above a threshold (e.g., median) level for that patient cohort; they are otherwise predicted to be ‘poor responders.’

The same cohorts were also stratified based on BRCA1/2 status alone. All modes of patient stratification were then compared to the actual clinical outcomes, using both log rank and COX proportional hazard ratio test statistics, to assess and quantify their predictive value. In each of the cohorts examined, the response outcomes predicted by the decision tree provided herein outperformed the predictive power of BRCA1/2 status alone, as indicated by lower hazard ratios and p-values (FIG. 6B).

The data provided herein support the hypothesis that ‘BRCAness status,’ and consequently HRD, are not good predictors of chemotherapeutic response per se, and that additional patient stratification taking into account how the BRCAness status is achieved in a cancer genome is a better predictor of patient responses. The data also highlight the relevance of BRCA1/2-based patient classification prior to assessing the potential role of immune infiltrates in guiding tumor responsiveness.

Some aspects of the present disclosure provide a method comprising: assaying a tumor sample from a subject for a wild-type BRCA1 and BRCA2 (BRCA1/2) gene mutational status; and assaying for an immune signature in the tumor sample identified as having a wild-type BRCA1/2 gene mutational status, wherein the immune signature is above or below a threshold level.

In some embodiments, the method further comprises selecting the subject for a therapy with a platinum-based agent if the immune signature is above the threshold level.

In some embodiments, the method further comprises selecting the subject for no therapy or a therapy other than the combination therapy if the immune signature is below a threshold level.

Other aspects of the present disclosure provide a method comprising: assaying a tumor sample from a subject for a BRCA1/2 gene mutational status selected from wild-type and mutated; optionally assaying the tumor sample identified as having a wild-type BRCA1/2 gene mutational status for an immune signature, wherein the immune signature is above or below a threshold level; and (a) selecting the subject for a therapy with a platinum-based agent if (i) the tumor sample has a wild-type BRCA1/2 gene mutational status and an immune signature above the threshold level, or (ii) the tumor has a mutated BRCA1/2 gene mutational status, or (b) selecting the subject for no therapy or a therapy other than a therapy with a platinum-based agent if the tumor sample has a wild-type BRCA1/2 gene mutational status and an immune signature below the threshold level.

Yet other aspects of the present disclosure provide a method comprising: assaying a tumor sample from a subject for a BRCA1/2 gene mutational status selected from wild-type and mutated; optionally assaying the tumor sample identified as having a wild-type BRCA1/2 gene mutational status for an immune signature, wherein the immune signature is above or below a threshold level; designating the subject as a good responder or a poor responder to a therapy with a platinum-based agent based on the BRCA1/2 gene mutational status of the tumor sample.

In some embodiments, the subject is designated as a good responder if the tumor sample has a mutated BRCA1/2 gene mutational status.

In some embodiments, the subject is designated as a good responder if the tumor sample has a wild-type BRCA1/2 gene mutational status and the immune signature is above the threshold level.

In some embodiments, the subject is designated as a poor responder if the tumor sample has a wild-type BRCA1/2 gene mutational status and the immune signature is below the threshold level.

In some embodiments, the method further comprises selecting the subject for a therapy with a platinum-based agent if the subject is a good responder.

In some embodiments, the immune signature is a M1 macrophage immune signature generated using an algorithm capable of estimating abundances of member cell types in a mixed cell population using gene expression data, optionally wherein the algorithm is CIBERSORT.

In some embodiments, the immune signature is an immune cell-specific enrichment score generated using an algorithm capable of calculating separate enrichment scores for each pairing of a sample and gene set, wherein each enrichment score represents the degree to which genes in a particular gene set are coordinately up-regulated or down-regulated within a sample, optionally wherein the algorithm is single sample Gene Set Enrichment Analysis (ssGSEA).

In some embodiments, the immune signature is an immune subtype assignment based on the TNBCtype classification system.

In some embodiments, the immune signature is based on the measurement of one or more immune gene set.

In some embodiments, the tumor sample is from a subject with breast cancer, optionally triple negative breast cancer (TNBC). In other embodiments, the tumor sample is from a subject with ovarian carcinoma (OV).

In some embodiments, the platinum-based agent is selected from the group consisting of oxaliplatin, cisplatin, carboplatin, nedaplatin, picoplatin, phenanthriplatin, triplatin, spiroplatin, satraplatin, iproplatin, and satraplatin.

In some embodiments, the therapy is a combination therapy that comprises at least one agent in addition to the platinum-based agent.

In some embodiments, the combination therapy comprises a taxane.

In some embodiments, the taxane is selected from the group consisting of paclitaxel, docetaxel, and cabazitaxel. In other embodiments, the combination therapy comprises cisplatin or carboplatin. In some embodiments, the combination therapy comprises docetaxel.

Other aspects of the present disclosure provide a method comprising assaying a primary tumor sample for promoter methylation status of a wild-type BRCA1 gene from a subject prior to the subject receiving a therapy; and (a) selecting the subject for a therapy with a platinum-based agent if the tumor sample has a promoter methylation status of fully methylated, or (b) selecting the subject for an alternative therapy without a platinum-based agent or no therapy if the tumor sample has a promoter methylation status of partially methylated or unmethylated.

Other aspects of the present disclosure provide a method comprising assaying a recurrent tumor sample for promoter methylation status of a wild-type BRCA1 gene from a subject, wherein a primary tumor sample from the subject was previously assayed for promoter methylation status of a wild-type BRCA1 gene, and wherein the promoter methylation status of the primary tumor was fully methylated; and (a) selecting the subject for a therapy with a platinum-based agent if the recurrent tumor sample has a promoter methylation status of fully methylated, or (b) selecting the subject for an alternative therapy without a platinum-based agent if the recurrent tumor sample has a promoter methylation status of partially methylated or unmethylated.

In some embodiments, the method further comprises assaying for expression of a wild-type BRCA1 gene.

In some embodiments, the promoter methylation status is assayed by Methylation-Specific PCR (MSP), Methylation-Specific Digital Droplet PCR (MS-ddPCR), and/or Next Generation Sequencing of bisulfite converted DNA amplicons.

In some embodiments, the tumor sample is from a subject with breast cancer, optionally triple negative breast cancer (TNBC).

In some embodiments, the tumor sample is from a subject with ovarian carcinoma (OV).

In some embodiments, the platinum-based agent is selected from the group consisting of oxaliplatin, cisplatin, carboplatin, nedaplatin, picoplatin, phenanthriplatin, triplatin, spiroplatin, satraplatin, iproplatin, and satraplatin.

In some embodiments, the therapy is a combination therapy that comprises at least one agent in addition to the platinum-based agent.

In some embodiments, the combination therapy comprises a taxane.

In some embodiments, the taxane is selected from the group consisting of paclitaxel, docetaxel, and cabazitaxel.

In some embodiments, the combination therapy comprises cisplatin or carboplatin.

In some embodiments, the combination therapy comprises docetaxel.

### BRIEF DESCRIPTION OF THE DRAWINGS

FIG. 1A provides an overview of the triple negative breast cancer (TNBC) cohort genetic, genomic and clinical features. Samples were sorted based on their tandem duplicator phenotype (TDP) group assignment. FIG. 1B provides data demonstrating an association between BRCA1 abrogation (by mutation or methylation) and TDP status (Type 1 TDP refers to TDP subgroups with short span tandem duplications (TDs) and comprises TDP groups 1, 1/2mix and 1/3mix). FIGS. 1C-1E provide data demonstrating associations between TDP status (FIG. 1C), BRCA1/2 mutation status (FIGS. 1D-1E), and BRCA1 methylation status (FIG. 1D) with pCR rates. FIG. 1F provides data demonstrating BRCA1 expression levels in BRCA1 methylated vs. mutated or wild type tumors. FIG. 1G provides data demonstrating the number of TDs across BRCA1 methylated, mutated and wild type genomes. FIG. 1H provides data demonstrating the TD span size distribution profiles across BRCA1 methylated, mutated and wild type genomes. A (*) indicates Student's t-test was used; a (**) indicates Mann-Whitney test was used.

FIG. 2A provides an overview of the genetic, genomic and clinical features relative to the two ovarian carcinoma cohorts analyzed. Samples were sorted based on their TDP group assignment. FIGS. 2B-2C provide data demonstrating an association between BRCA1 abrogation (by mutation or methylation) and TDP status in the AOCS dataset (FIG. 2B) and in the in-house OvCa dataset (FIG. 2C). FIG. 2D provides data demonstrating the therapy response in the Australian Ovarian Cancer Study (AOCS) dataset, with patients grouped based on BRCA1/2 status and on TDP status. Logistic regression was used to compute p-values. FIG. 2E provides data demonstrating overall survival curves relative to the AOCS dataset, with patients grouped based BRCA1/2 status and TDP status. Curve comparison between the different subgroups was performed with the log-rank test and statistically significant differences are shown on graph. FIG. 2F provides data demonstrating the therapy response in the in-house OvCa dataset, with patients grouped based on BRCA1/2 status and on TDP status. Logistic regression was used to compute p-values. FIG. 2G provides data demonstrating disease-specific survival curves relative to the in-house OvCa dataset, with patients grouped based on BRCA1/2 status and TDP status and optimal debulk. Curve comparison between the different subgroups was performed with the log-rank test and statistically significant differences are shown on graph.

FIG. 3A provides an overview of the genetic, genomic and response features relative to PDX cohort. Samples are sorted based on their TDP group assignment. FIGS. 3B-3D provide data demonstrating TDP features and therapeutic response across a cohort of TNBC PDX models. FIG. 3B provides data demonstrating TDP features (BRCA1/2 wildtype, BRCA1/2 mutant, and BRCA1 methylated) and therapeutic response. FIG. 3C provides data demonstrating TDP features (Non TDP, TDP type 1, TDP other) and therapeutic response. FIG. 3D provides data demonstrating TDP features (BRCA1/2 wildtype, BRCA1/2 mutant, and BRCA1 methylated) and therapeutic response to Docetaxel.

FIGS. 4A-4E provide data demonstrating measures of HRD and therapeutic response in TNBC and ovarian cancer. FIG. 4A provides data demonstrating similar TD span size distributions across tumors stratified based on their BRCA1 status. P-values by Mann-Whitney test. FIG. 4B provides data demonstrating TDP score distributions across tumors stratified based on their BRCA1 status. P-values by Mann-Whitney test. FIG. 4C provides data demonstrating similar HRDetect scores across a meta-cohort of 877 breast and ovarian cancer genomes stratified based on their BRCA1/2 status. P-values by Mann-Whitney test. FIG. 4D provides data demonstrating overall survival outcomes in a subset of the AOCS dataset stratified based on BRCA1/2 status, HRDetect score and TDP status. Hazard ratios (HRs) are computed based on three different modes of patient stratification. HR and P-values by coxph. FIG. 4E provides data demonstrating overall survival of OvCa patients with BRCA1/2 wild type tumors as a function of the HRDetect score.

FIG. 5A provides data in the form of barplots demonstrating TNBC type classification based on the six transcriptional subtypes. The number of TNBCs corresponding to the immunomodulatory subtype was compared between responders (i.e., pCR) vs. non-responders (i.e., SD/PR) using the Fisher's exact test. Unclassified tumors are depicted at the top of the plots were not included in the statistical analysis. FIG. 5B provides data in the form of volcano plots demonstrating differential gene expression between responder (pCR) and non-responder (PR/SD) patients within the TNBC cohort. Significantly differentially expressed genes (p-value<0.05 and absolute log 2 fold change>1), are indicated by (DOWN) and (UP) boxes. Enrichment of immune genes within the up-regulated differentially expressed genes was computed by Fisher's exact test. FIG. 5C provides CIBERSORT scores that were compared between responder and non-responder subgroups for each independent tumor cohort, using the Student's t.test and only significant p.values are reported in the figure. FIGS. 5D-5E provide data in the form of Kaplan-Meier curves demonstrating overall survival for patients with high and low levels of the CIBERSORT-derived macrophages M1 gene signature scores (split on the median level for each indicated group).

FIG. 6A provides a schematic of the proposed decision tree for optimal prediction of TNBC and OV patient response to platinum-taxane combination chemotherapy. M1 refers to the M1 macrophage signal computed using CIBERSORT, with high and low defined based on the median score value for all samples in the BRCA1/2 wild type tumor cohort. FIG. 6B provides data demonstrating hazard ratios for overall survival of OV patients for the in-house OV dataset (left) and for the AOCS dataset (right), stratified based on either BRCA1/2 status alone, or a combination of BRCA1/2 status and relative score for the M1 macrophage immune signature (computed as described in FIG. 6A). Only significant p-values are reported (COX proportional hazard test).

FIG. 7A provides data in the form of Kaplan-Meier curves demonstrating the survival of the indicated subgroups of OvCa patients; of note: only patients who died of the disease were included in the analysis (n=65). FIG. 7B provides data in the form of Kaplan-Meier curves demonstrating the survival of the subset of OvCa patients that did not received optimal debulk, sub-grouped based on their BRCA1/2 status. Curve comparison between the different subgroups was performed with the log-rank test and statistically significant differences are shown on graph. A (*) indicates P<0.05.

FIG. 8 provides data demonstrating the response outcome in OvCa patients stratified by both BRCA1/2 status and HRDetect score.

FIG. 9A provides data demonstrating that TOP 20 GO biological process terms were significantly enriched in the set of up-regulated genes in TNBC of responders vs. non-responders. Terms were sorted by increasing p-value and their relative fold enrichment is indicated on the x axis. The number of significant genes for each category is depicted within each bar. FIG. 9B provides data in the form of a volcano plot demonstrating differential gene expression between responder (pCR) and non-responder (PR/SD) patients within the TNBC cohort. Significantly differentially expressed genes (p-value<0.05 and absolute log 2 fold change >1), are indicated by (DOWN) and (UP) boxes. Enrichment of immune genes within the up-regulated differentially expressed genes was computed by Fisher's exact test.

FIG. 10A provides scores representing enrichment values computed using the ssGSEA algorithm. Responder and non-responder subgroups were compared using the Student's t-test and only significant p-values are reported in the figure. Boxes indicate the M1 macrophage gene set. FIGS. 10B-10C provide data in the form of Kaplan-Meier curves demonstrating overall survival for patients with high and low levels of the ssGSEA-derived macrophages M1 gene enrichment scores (split on median level within each indicated group).

FIG. 11 provides data demonstrating a comparison of immune gene set scores between chemotherapy responders and non-responders across the BRCA1/2 mutant tumor patient population.

FIG. 12 provides data demonstrating a comparison of immune gene set scores between chemotherapy responders and non-responders across BRCA1 methylated tumor patient populations.

FIGS. 13A-13B provide data in the form of Kaplan-Meier curves for OV patients in the in-house OV dataset (FIG. 13A) and in the AOCS dataset (FIG. 13B), stratified based on either BRCA1/2 status alone (left), or a combination of BRCA1/2 status and relative score for the M1 macrophage immune signature (right, as described in FIG. 6A). Log-rank test and COX proportional hazard test statistics are reported in the corresponding tables.

FIGS. 14A-14H provide patterns of BRCA1 methylation and chemotherapy response in TNBC PDXs. FIG. 14A provide data of BRCA1 gene expression (qpCR) as a function of BRCA1 status in TNBC PDX models. The average value of all the assessed BRCA1 wild type PDX models was used as the calibrator in assessing expression fold changes. P values by Student's t-test. FIG. 14B provides data of BRCA1 methylation status and tumor source for a set of 12 BRCA1 methylated PDX models of TNBC. P value by Fisher's Exact test. FIG. 14C provides a description of the tumor origin for the three PDX models established from the same TNBC patient donor. FIG. 14D provides MSP results for the three PDX models in the single patient donor longitudinal series. FIG. 14E provides BRCA1 expression levels for the three PDX models in a longitudinal series, assed by qPCR. A BRCA1 wild type PDX tumor is used as the reference sample. FIG. 14F provides BRCA1 methylation status and platinum-based therapy response for a set of 16 BRCA1 methylated PDX models of TNBC. Identical outcomes were obtained when response was determined using RECIST criteria, as reported in Table S5. P value by Fisher's Exact test. FIG. 14G provides MSP results for vehicle tumors and cisplatin-treated tumor recurrences relative to PDX #TM00097. FIG. 14H provides qPCR results of BRCA1 gene expression across vehicle tumors and cisplatin- or docetaxel-treated tumor recurrences relative to five TNBC PDX models with different BRCA1/2 backgrounds. All the data is normalized to the average BRCA1 expression level of control tumors from the BRCA1/2 wild type PDX #TM00096. P values by Student's t-test.

FIGS. 15A-15J provide BRCA1 methylation and chemotherapy response in PDX and primary OvCa cohorts. FIG. 15A provides MSP results for three exemplary TNBC PDX models, showing three different patterns of BRCA1 methylation at the proximal promoter. U, unmethylated PCR product, M, methylated PCR product. FIG. 15B provides tumor growth curves for the three TNBC PDX models of the longitudinal series. Animals in the carboplatin arm were dosed weekly for four consecutive weeks. The reported value on each graph corresponds to the average percentage reduction in tumor growth rate for the carboplatin arm compared to the control arm. Responders are identified as PDX models showing a >5% growth rate reduction. FIG. 15C provides tumor growth curves relative to TNBC PDX #TM00097. Black arrow heads indicate the timing of the four weekly doses of cisplatin. FIG. 15D provides tumor growth curves relative to TNBC PDX #TM01097. Black arrow heads indicate weekly doses of cisplatin. A red arrow indicates the growth curve for animal #097-01, whose tumor maintained a full methylation profile, as assessed in FIG. 15E. FIG. 15E provides MSP results for vehicle tumors and cisplatin-treated tumor recurrences relative to PDX #TM01079. FIG. 15F provides a correlation between BRCA1 gene expression (RNAseq log 2 values) and BRCA1 promoter methylation (based on MS-ddPCR estimates corrected for the proportion of neoplastic cellularity). Data relative to the subset of 13 BRCA1 methylated UW OvCas with available expression data is shown. FIG. 15G provides BRCA1 gene expression (RNAseq log 2 values) across the UW OvCa patient cohort subgrouped based on BRCA1 status. P value by Student's t-test. FIG. 15H provides BRCA1 methylation status (via MS-ddPCR) and five-year survivorship for the subset of BRCA1 methylated patients who received optimal debulking in the UW OvCa cohort. Patients with less than 5 years of follow up are not depicted. FIG. 15I provides a summary of structural variations and tandem duplication span sizes relative to the paired primary and recurrent OvCa genomes from donor AOCS-091 (AOCS cohort). FIG. 15J provides MSP results for nine single cell-derived clonal expansions of the primary cultures established from the BRCA1 partially methylated TNBC PDX #TM00099.

### DETAILED DESCRIPTION

The present disclosure provides, in some aspects, methods for predicting a cancer (e.g., breast cancer or ovarian cancer) patient's response to platinum-based chemotherapy. The experimental data provided herein was generated using (a) a detailed analysis of whole genome sequence (WGS) data, (b) treatment response studies from three cohorts of patients having triple negative breast cancer (TNBC) or ovarian cancer who were treated with chemotherapeutic agents containing a platinum-based compound, and (c) TNBC patient-derived xenograft (PDX) models, collectively demonstrating that BRCA1 and BRCA2 (BRCA1/2) pathogenic mutations in certain patient populations are associated with positive responses to platinum-based chemotherapy. The data provided herein also suggests that the previously used classification of tandem duplicator phenotype (TDP) score or TDP type does not predict for improved chemotherapeutic response and further suggests that the general phenotype of genomic instability as measured by any instability score such as LOH-based HRD metrics may be insufficient to predict for therapeutic response. The data provided herein highlights a clear differential effect on therapeutic response between tumors harboring BRCA1 mutations versus those with reduced BRCA1 expression through promoter methylation, even though the two forms of BRCA1 alterations results in identical genomic scarring. The present disclosure provides data supporting an association between immune score and therapeutic response in TNBC and ovarian cancer appear to be most predictive in the BRCA1/2 wildtype subgroup. Further, the present disclosure provides data provided supporting full BRCA1 promoter methylation in primary and/or recurrent tumors is predictive of positive therapeutic response to platinum-based agents.

### BRCA1/2 Mutational Status

Breast cancer early onset gene 1 (BRCA1) is a tumor suppressor gene located on chromosome 17q21 that encodes a protein composed of 1863 amino acid residues. BRCA1 regulates transcription, DNA double-strand breaks, and recombination. Breast cancer early onset gene 2 (BRCA2) is a tumor suppressor gene located on chromosome 13q12 that encodes a protein composed of 3418 amino acids. Both BRCA1 and BRCA2 are homology-directed repair (HDR) genes and are associated with predispositions to breast, ovarian, and, at lower frequency, prostate, pancreatic, and other cancers. As used herein, the term BRCA1/2 refers to BRCA1 and BRCA2.

The methods described herein, in some embodiments, comprise assaying a tumor sample for a BRCA1/2 gene mutational status. The BRCA1/2 gene mutational status of a tumor sample corresponds to the BRCA1/2 gene mutational status of the BRCA1/2 genes in the tumor sample. The term BRCA1/2 gene mutational status refers to one of two BRCA1/2 genotypes: wild-type BRCA1/2 and mutated BRCA1/2. BRCA1/2 gene mutational status can be determined by any method used for genotyping and/or method for determining epigenetic changes (e.g., methylation) in a gene such as, for example, Sanger sequencing/multiplex ligation-dependent probe amplification (MLPA) and next-generation sequencing (NGS). The term wild-type BRCA1/2 gene mutational status, as used herein, is assigned to BRCA1/2 genes that encode functional proteins (e.g., protein activities capable of regulating transcription, DNA double-strand breaks, and/or recombination). It should be understood that BRCA1/2 genes assigned to a wild-type BRCA1/2 ‘mutational status’ need not be wild-type genes in the conventional sense but may include a (at least one) modification (e.g., missense mutation and/or single nucleotide polymorphism), relative to the BRCA1 and BRCA2 genes having, for example, the nucleic acid sequence listed in the Ensemble database at ENSG00000012048 and ENSG00000139618, respectively, provided the BRCA1/2 genes encode functional proteins.

In some embodiments, BRCA1/2 genes assigned to a wild-type BRCA1/2 mutational status encode proteins that exhibit a level of activity within 50% (e.g., within 45%, 40%, 35%, 30%, 25%, 20%, 15%, 10%, 5%) of the level of activity of a protein encoded by BRCA1/2 genes comprising the nucleic acid sequence listed in the Ensemble database at ENSG00000012048 and ENSG00000139618, respectively. BRCA1 protein activity may be measured using, for example, the transcription activation (TA) assay (Monteiro et al., 1998, incorporated by reference), the small colony phenotype assay (Coyneh et al., 2004, incorporated by reference), the rescue of radiation sensitivity assay (Scully et al., 1999, incorporated by reference), the ubiquitin ligase activity assay (Morris et al., 2006, incorporated by reference), or functional complementation assays, and PARP inhibitor sensitivity assay (Bouwman et al, 2013, incorporated by reference). Other BRCA1 functional assays known or later developed are also contemplated. BRCA2 protein activity may be measured using, for example, in vitro assays that measure (a) mitomycin C (MMC) sensitivity, (b) homologous recombination DNA repair and (c) centrosome amplification (Wu et al., 2005, incorporated by reference). Other BRCA2 functional assays known or later developed are also contemplated.

The term mutated BRCA1/2 gene mutational status, as used herein, is assigned to BRCA1/2 loss-of-function alleles. Loss-of-function alleles, as in known in the art, include complete loss-of-function alleles (alleles that do not encode a functional protein—also known as amorph or null alleles) as well as partial loss-of-function alleles (alleles that encode an incompletely functioning protein—also known as hypomorphs or leaky mutations). BRCA1/2 loss-of-function alleles may include any pathogenic or likely pathogenic mutation. A pathogenic mutation is any genetic alteration that increases a subject's susceptibility or predisposition to a certain disease or disorder, such as cancer (e.g., breast cancer or ovarian cancer). Some types of BRCA1/2 mutations are known in the art to be pathogenic. Other types of BRCA1/2 mutations, which are predicted by those in the art to be pathogenic, are termed “likely pathogenic” mutations. In some embodiments, BRCA1/2 mutations are classified as pathogenic or likely pathogenic based on a mutation database such as the ClinVar database (www.ncbi.nlm.nih.gov/clinvar/, incorporated by reference), and/or the ARUP BRCA database (https://arup.utah.edu/database/BRCA1, incorporated by reference). Other databases that may be used by a those skilled in the art to determine whether a mutation is pathogenic or likely pathogenic are also contemplated. In some embodiments, a tumor sample is assigned a mutated BRCA1/2 gene mutational status if the tumor sample comprises BRCA1/2 genes that have pathogenic or likely pathogenic mutations based on a mutation database described above or otherwise known in the art.

Pathogenic mutations may emerge in various forms including, but not limited to, nonsense mutations, frameshift mutations, missense mutations, short insertions/deletions, splice site mutations, stop codon read-through mutations and rearrangements). In some embodiments, BRCA1/2 pathogenic mutations include nonsense or missense mutations. In some embodiments, BRCA1/2 pathogenic mutations include mutations that result in a frameshift of the resulting amino acid sequence. In some embodiments, BRCA1/2 pathogenic mutations include mutations that result in short insertion/deletion sequences within the BRCA1/2 exon regions. In some embodiments, BRCA1/2 pathogenic mutations include mutations that result in changes to the splice site of the BRCA1/2 exons. In some embodiments, BRCA1/2 pathogenic mutations include mutations that result in premature stop codons. In some embodiments, BRCA1/2 pathogenic mutations may be a structural rearrangement of BRCA1/2 gene regions, such as a structural rearrangement that results in the loss or translocation of at least part of the BRCA1/2 gene coding region.

In some embodiments, BRCA1/2 genes assigned to a mutant BRCA1/2 mutational status encode proteins that exhibit a level of activity that is less than 50% (e.g., less than 45%, 40%, 35%, 30%, 25%, 20%, 15%, 10%, or 5%) of the level of activity of a protein encoded by BRCA1/2 genes comprising the nucleic acid sequence listed in the Ensemble database at ENSG00000012048 and ENSG00000139618, respectively.

In some embodiments, BRCA1/2 genes assigned to a mutant BRCA1/2 mutational status encode nonfunctional proteins (no detectable function).

In some embodiments of the present disclosure, the BRCA1/2 gene mutational status of a tumor sample is from a subject with triple negative breast cancer (TNBC). In some embodiments of the present disclosure, the BRCA1/2 gene mutational status of a tumor sample is from a subject with ovarian carcinoma (OV).

### BRCA1 Promoter Methylation

The methods described herein, in some embodiments, comprise assaying a tumor sample for BRCA1 promoter methylation status. Promoter methylation is a form of epigenetic regulation wherein methyl groups added to DNA molecules may alter the activity of gene expression without altering the DNA sequence. In some embodiments, BRCA1 promoter methylation may act to repress gene transcription of the BRCA1 gene and decrease BRCA1 protein expression. In some embodiments, a decrease in BRCA1 promoter methylation may result in increased BRCA1 protein expression. In some embodiments, a tumor sample is assayed for BRCA1 promoter methylation status. In some embodiments, a tumor sample is assayed for both BRCA1 promoter methylation status and BRCA1 protein expression.

Promoter methylation may be measured or determined using a variety of methods known in the art, including but not limited to, Methylation-Specific PCR (MSP), Methylation-Specific Droplet Digital PCT (MS-ddPCR), and/or Next Generation Sequencing of bisulfite converted DNA amplicons. Other methods of quantifying promoter methylation known in the art are also contemplated herein.

The term BRCA1 promoter methylation status refers to the level of methylation of the DNA within the promoter region of the BRCA1 gene measured in a tumor sample from a subject (e.g., BRCA1 promoter methylation status can be fully methylated, partially methylated, or unmethylated). In some embodiments, the BRCA1 promoter methylation status of a primary or recurrent tumor from a subject may inform therapy selection.

In some embodiments, a primary tumor sample from a subject comprises a BRCA1 promoter methylation status that is fully methylated. In some embodiments, a subject may respond better to therapy with a platinum-based agent if a primary tumor sample from the subject has a promoter methylation status of fully methylated. In some embodiments, a subject may be selected for therapy with a platinum-based agent if a primary tumor sample from the subject has a promoter methylation status of fully methylated.

In some embodiments, a primary tumor sample from a subject comprises a BRCA1 promoter methylation status that is partially methylated or unmethylated. In some embodiments, a subject may respond better to an alternative therapy that does not include a platinum-based agent if a primary tumor sample from the subject has a promoter methylation status of partially methylated or unmethylated. In some embodiments, a subject may be selected for an alternative therapy that does not include a platinum-based agent if a primary tumor sample from the subject has a promoter methylation status of partially methylated or unmethylated.

In some embodiments, a recurrent tumor sample from a subject comprises a BRCA1 promoter methylation status that is fully methylated. In some embodiments, a subject may respond better to therapy with a platinum-based agent if a recurrent tumor sample from the subject has a promoter methylation status of fully methylated. In some embodiments, a subject may be selected for therapy with a platinum-based agent if a recurrent tumor sample from the subject has a promoter methylation status of fully methylated.

In some embodiments, a recurrent tumor sample from a subject comprises a BRCA1 promoter methylation status that is partially methylated or unmethylated. In some embodiments, a subject may respond better to an alternative therapy that does not include a platinum-based agent if a recurrent tumor sample from the subject has a promoter methylation status of partially methylated or unmethylated. In some embodiments, a subject may be selected for an alternative therapy that does not include a platinum-based agent if a recurrent tumor sample from the subject has a promoter methylation status of partially methylated or unmethylated.

### Immune Signature

The methods described herein, in some embodiments, comprise assaying a tumor sample for an immune signature. The term immune signature refers to a distinct molecular profile of a tumor (or tumor sample), taking into account the types of immune cells infiltrating the tumor (referred to as immune infiltrate), as well as the gene and/or protein expression profile of cells (resident or infiltrate) in the tumor (or tumor sample).

In some embodiments, an immune signature includes a gene expression signature, which may be determined, for example, by assessing DNA and/or RNA expression patterns and/or levels in a tumor (or tumor sample). Non-limiting examples of assays for assessing gene expression patterns and/or levels include RNA-SEQ, target capture RNAseq, microarray, quantitative PCR.

In some embodiments, an immune signature includes a protein expression and/or activity signature, which may be determined, for example, by assessing protein expression patterns, levels, and/or activity in a tumor (or tumor sample). Non-limiting examples of assays for assessing protein expression patterns, levels, and/or activity include immunohistochemical assays, immunocytochemical assays and Western Blotting.

In some embodiments, an immune signature includes an immune infiltrate signature, which may be determined, for example, by assessing, directly or indirectly, the types of immune cells present in a tumor (or tumor sample). Non-limiting examples of assays for assessing immune infiltrate include the gene and protein expression assays above as well as single cell RNAseq, CITE-seq and NanoString Technologies gene expression assays. Examples of cells that may be present in an immune infiltrate include lymphoid cells, such as cytotoxic T lymphocytes (CD3⁺·CD8⁺), regulatory T lymphocytes (CD3⁺, CD4⁺, CD25⁺, FOXP3⁺), T helper lymphocytes (CD4⁺) and natural killer cells (CD16⁺, CD56⁺), as well as myeloid cells, such as dendritic cells (CD40⁺), myeloid-derived suppressor cells (MDSC) (CD11b⁺, CD66b⁺) and macrophages (CD68⁺). In some embodiments, the following cell surface markers are used to assay an immune infiltrate of a tumor sample: PD-1, PD-L1, CD3, CD4, CD8, CD25, IFNγ, LAG3, and FoxP3. In some embodiments, an immune signature of the present invention refers to an immune infiltrate of a tumor sample. In some embodiments, an immune infiltrate may be a subset of an immune signature.

An immune signature may be assessed using one or more of a variety of different assays and algorithms known or later developed. For example, an immune signature may be generated using an algorithm capable of estimating abundances of member cell types in a mixed cell population using gene expression data (e.g., RNA-SEQ data). The output of such an algorithm is an abundance score for member cell types. The abundance score may be based on all immune cell types or a subset of immune cell subtypes, for example. In some embodiments, the algorithm used to estimate abundance of member cell types in a mixed cell population is trained on all immune cell subtypes (e.g., lymphoid cells and/or myeloid cells). In some embodiments, the algorithm used to estimate abundance of member cell types in a mixed cell population is trained on a subset of immune cell subtypes. Non-limiting examples of algorithms that could be used are associated with the following computational tools: the Microenvironment Cell Populations-Counter (MCP-counter) (Becht et al., 2016, incorporated by reference herein), the University of San Francisco xCell webtool, the Tumor Immune Estimation Resource (TIMER), and CIBERSORT (Chen et al. 2018, incorporated by reference herein).

In some embodiments, an immune signature of a tumor sample can be assayed by generating a tumor sample CIBERSORT score of a (at least one) immune cell-specific gene expression profile. In some embodiments, one immune cell-specific expression profile is used to generate a CIBERSORT score of a tumor sample. In some embodiments, two immune cell-specific expression profiles are used to generate two CIBERSORT scores of a tumor sample and the two CIBERSORT scores are considered together. In some embodiments, a CIBERSORT score is a M1 macrophage signal in a tumor sample.

An immune signature may also be assayed using a computational method that determines whether an a priori defined set of genes (e.g., gene sets) shows statistically significant, concordant differences between two biological states (e.g., tumor phenotype relative to normal phenotype). This computational method may be, for example, a Gene Set Enrichment Analysis (GSEA), wherein a gene set's enrichment score is generated with respect to phenotypic differences across a collection of a samples within a dataset. Many tools are available for performing a GSEA computational method, such as, Nucleic Acid SeQuence Analysis Resource (NASQAR), PlantRegMap, Molecular Signature Database (MSigDB), Broad Institute downloadable GSEA software, WebGestalt (a web based gene set analysis toolkit), Enrichr (a gene set enrichment analysis tool for mammalian gene sets), GeneSCF, the Database for Annotation, Visualization and Integrated Discovery (DAVID), Metascape, AmiGO 2 (a gene ontology enrichment tool), Genomic region enrichment of annotations tool (GREAT), the Functional Enrichment Analysis (FunRich), FuncAssociate (tool that enables gene ontology and custom enrichment analyses), InterMine, ToppGene Suite, Quantitative Set Analysis for Gene Expression (QuSAGE), Blast2GO, and g:Profiler. Other tools for performing a GSEA computation method known to those skilled in the art are also contemplated. In some embodiments, an immune signature of a tumor sample is assayed by an immune cell-specific enrichment score computed via a GSEA tool known or later developed.

The computational method may be, for example, a single-sample Gene Set Enrichment Analysis (ssGSEA), wherein a separate enrichment score for each pairing of sample and gene set, independent of phenotype labeling is generated. The ssGSEA computational method transforms a single sample's gene expression profile to a gene set enrichment profile. A gene set's enrichment score represents the activity level of the biological process in which the gene set's members are coordinately up-regulated or down-regulated. This transformation permits the cell state of a sample, such as a tumor sample, to be characterized in terms of the activity levels of biological processes and pathways rather than through the expression levels of individual genes. In some embodiments, an immune signature of a tumor sample is assayed by an immune cell-specific enrichment score computed via a ssGSEA tool known or later developed.

In some embodiments, an immune signature is assayed by using an immune cell-specific enrichment score computed via a ssGSEA immune-related gene set defined by Barbie et al. 2009, incorporated by reference herein. Such immune-related gene sets can be found, for example, at Molecular Signatures Database, (broad.mit.edu/gsea/msigdb/).

In some embodiments, an immune signature is assayed using immune gene set enrichment-based methods defined by Davoli et al. 2017, which is incorporated by reference herein. Such immune-related gene sets, which defined gene expression for immune cell types (e.g., CD8+ T cells, B cells, NK cells, Tregs, CD4+ T cells, dendritic cells, and macrophages) can be found, for example, at the Immunological Genome Project (ImmGen) database.

In some embodiments, the immune signature of the present disclosure is assayed by other immune-related gene set enrichment-based methods known or later developed.

In some embodiments, an immune signature can be determined using an immunomodulatory subtype assignment based on the TNBCtype classification system described by Chen at al. 2012, incorporated by reference herein. As used herein, the TNBCtype classification system refers to six triple negative breast cancer subtypes (e.g., basal-like 1 (BL1), basal-like 2 (BL2), immunomodulatory (IM), mesenchymal (M), mesenchymal stem-like (MSL), and luminal androgen receptor (LAR)) identified by clustering analysis of 14 publicly available microarray data-sets. The immunomodulatory (IM) subtype includes canonical pathways such as the CTLA4 pathway, the IL12 pathway, the NK pathway, the Th1/Th2 pathway, the IL7 pathway, the antigen processing/presentation pathway, the NF-kB pathway, the TNF pathway, the T cell signal transduction pathway, the DC pathway, the BCR signaling pathway, the NK cell mediated cytotoxicity pathway, the JAK/STAT signaling pathway, and the ATR/BRCA pathway. In some embodiments, the immune signature of a tumor sample is assayed using the immunomodulatory subtype assignment based on the TNBCtype classification system described by Chen et al., 2012.

Other methods for assaying an immune signature of a tumor sample known to one of ordinary skill in the art are also contemplated.

The methods provided herein, in some embodiments, include comparing an immune signature to a threshold value. A threshold value is associated with a statistic. As used herein, it is a value above or below which an immune signature is assigned significance, in particular with respect to a subject's responsiveness to therapy with a platinum-based agent. See Examples section below. In some embodiments, a threshold value is a median value. In some embodiments, a threshold value is an average value. In some embodiments, a threshold value is a mean value.

In some embodiments provided herein, a subject is selected for a therapy with a platinum-based agent if a tumor sample has a wild-type BRCA1/2 gene mutational status and an immune signature above the threshold level. In other embodiments, a subject is selected for a therapy with a platinum-based agent if the tumor has a mutated BRCA1/2 gene mutational status. In yet other embodiments, a subject is selected for no therapy or a therapy other than a therapy with a platinum-based agent if the tumor sample has a wild-type BRCA1/2 gene mutational status and an immune signature below the threshold level.

### Prognostic Methods

The present disclosure provides a method for measuring the BRCA1/2 gene mutational status of a tumor sample from a subject. In some embodiments, the method further provides for categorizing the status of a subject with one of the following: a mutated BRCA1/2 gene mutational status or a wild-type BRCA1/2 gene mutational status.

In some embodiments, the method further identifies a subject as a candidate for platinum-based therapy based on the BRCA1/2 gene mutational status of their tumor sample. As used herein, “candidate” refers to a subject that may be responsive to a proposed treatment. As used herein, “platinum-based therapy” refers to administration of a platinum-based agent (i.e., a platinum-based therapeutic agent) to selectively kill or inhibit the growth, proliferation, and division of tumor cells.

The present disclosure provides, in some embodiments, the method for administering the platinum-based therapy to the subject based on the BRCA1/2 gene mutational status of the tumor sample from the subject. Administering can be by any method known in the art. Non-limiting examples of administering include intravenous, intraarterial, inhalation, ingestion of solid, ingestion of liquid, intradermal, intranasal, intramucosal, intraocular, intracranial, or intrathecal.

In some embodiments, a subject of the present disclosure is further categorized by how the subject is predicted to respond to the therapy. For example, a subject may be predicted to be a “good responder” or a “poor responder” to therapy based on the categorization of the BRCA1/2 gene mutational status of the tumor sample. As used herein, a good responder refers to a subject wherein the tumor may respond to treatment as indicated by a decrease in tumor size, an absence of secondary site tumors/metastases, and/or increased overall survival (OS) of the subject. As used herein, a poor responder refers to a subject wherein the tumor does not respond to treatment as indicated by the absence of a decrease in tumor size, the increase in tumor size, the presence of secondary site tumors/metastases, and/or an absence of increase in OS of the subject or a decrease in OS of the subject.

In some embodiments, a subject comprising a tumor with a mutated BRCA1/2 gene mutational status is predicted to be a good responder to the therapeutic agent. In some embodiments, the subject comprising a tumor with a mutated BRCA1/2 gene mutational status is selected for therapy with a platinum-based agent. In some embodiments, a platinum-based agent is administered to the subject with mutated BRCA1/2 gene mutational status.

In some embodiments, a subject comprising a wild-type BRCA1/2 gene mutational status tumor sample is further assayed for an immune signature above or below a determined threshold level. In some embodiments, wherein the immune signature is above the threshold level, the subject comprising a wild-type BRCA1/2 gene mutational status is selected for therapy with a platinum-based agent. In some embodiments, the subject is predicted to be a good responder to the therapeutic agent. In some embodiments, a platinum-based agent is administered to the subject with a wild-type BRCA1/2 gene mutational status wherein the immune signature is above the threshold level. In some embodiments, wherein the immune signature is below the threshold level, the subject is selected for no therapy or a therapy other than a therapy with a platinum-based agent. In some embodiments, the subject comprising a wild-type BRCA1/2 gene mutational status is predicted to be a poor responder to the therapeutic agent. In some embodiments, a platinum-based agent is not administered to the subject with a wild-type BRCA1/2 gene mutational status wherein the immune signature is below the threshold level.

### Platinum-Based Therapy

Platinum-based agents contain a platinum molecule conjugated to organic molecules including amines (NH2), amides (NH3), and chlorides (Cl). Platinum-based agents are effective at killing tumor cells because they are conjugated to DNA and inhibit DNA transcription, replication and repair. Non-limiting examples of platinum-based agents includeoxaliplatin, cisplatin, carboplatin, nedaplatin, picoplatin, phenanthriplatin, triplatin, spiroplatin, satraplatin, iproplatin, and satraplatin. In certain embodiments, the platinum-based therapeutic agent is any one described in Apps et al., The state-of-play and future of platinum drugs. Endocrine-Related Cancer 22:R219-R233, 2015 (incorporated herein by reference).

In some embodiments, a platinum-based therapy for a subject with a mutated BRCA1/2 gene mutational status, a wild-type BRCA1/2 gene mutational status, or a BRCA1 gene methylation status is a single platinum-based agent.

In some embodiments, a platinum-based therapy for a subject with mutated BRCA1/2 gene mutational status, wild-type BRCA1/2 gene mutational status or BRCA1 gene methylation status is a platinum-based therapy, wherein a single platinum-based agent is selected from the group consisting of oxaliplatin, cisplatin, carboplatin, nedaplatin, picoplatin, phenanthriplatin, triplatin, spiroplatin, satraplatin, iproplatin, and satraplatin.

In some embodiments, a platinum-based therapy for a subject with a mutated BRCA1/2 gene mutational status, a wild-type BRCA1/2 gene mutational status or a BRCA1 gene methylation status is a single platinum-based agent and/or a combination of platinum-based agents, or at least one platinum-based agent in combination with at least one other agent (e.g., a combination therapy). For example, other agents may include alkylating agents (e.g., cyclophosphamide, mechlorethamine, chlorambucil, melphalan, dacarbazine, nitroureas, temozolomide), anthracyclines (e.g., daunorubicin, doxorubicin, epirubucin, idarubicin, mitoxantrone, valrubicin), taxanes (e.g., paclitaxel, docetaxel, cabazitaxel, abraxane, taxotere), histone deacetylase inhibitors (e.g., vorinostat and romidepsin), topoisomerase inhibitors (e.g., irinotecan, topotecan, etoposide, teniposide, tafluposide), kinase inhibitors (e.g., bortezomib, erlotinib, gefitinib, imatinib, vemurafenib, vismodegib), nucleotide analogs (e.g., azacitidine, azathioprine, capecitabine, cytarabine, doxifluridine, fluorouracil, gemcitabine, hydroxyurea, mercaptopurine, methotrexate, tioguanine), retinoids (e.g., tretinoin, alitretinoin, bexarotene), and vinca alkaloids and derivatives (e.g., vinblastine, vincristine, vindesine, vinorelbine).

In some embodiments, a platinum-based therapy for a subject with mutated BRCA1/2 gene mutational status, wild-type BRCA1/2 gene mutational status or BRCA1 gene methylation status is a combination platinum-based therapy wherein at least two platinum-based agents are selected from the group consisting of oxaliplatin, cisplatin, carboplatin, nedaplatin, picoplatin, phenanthriplatin, triplatin, spiroplatin, satraplatin, iproplatin, and satraplatin.

In some embodiments, a platinum-based therapy for a subject with mutated BRCA1/2 gene mutational status, wild-type BRCA1/2 gene mutational status or BRCA1 gene methylation status is a combination platinum-based therapy comprises one or more platinum-based agents selected from the group consisting of oxaliplatin, cisplatin, carboplatin, nedaplatin, picoplatin, phenanthriplatin, triplatin, spiroplatin, satraplatin, iproplatin, and satraplatin, and a taxane. In some embodiments, the combination therapy comprises cisplatin or carboplatin. In some embodiments, the taxane is selected from the group consisting of paclitaxel, docetaxel, and cabazitaxel.

### Additional Embodiments

Additional embodiments of the present disclosure are encompassed by the following numbered paragraphs:
1. A method comprising: assaying a tumor sample from a subject for a wild-type BRCA1 and BRCA2 (BRCA1/2) gene mutational status; and assaying for an immune signature in the tumor sample identified as having a wild-type BRCA1/2 gene mutational status, wherein the immune signature is above or below a threshold level.
2. The method of paragraph1 further comprising selecting the subject for a therapy with a platinum-based agent if the immune signature is above the threshold level.
3. The method of paragraph1 further comprising selecting the subject for no therapy or a therapy other than the combination therapy if the immune signature is below a threshold level.
4. A method comprising: assaying a tumor sample from a subject for a BRCA1/2 gene mutational status selected from wild-type and mutated; optionally assaying the tumor sample identified as having a wild-type BRCA1/2 gene mutational status for an immune signature, wherein the immune signature is above or below a threshold level; and (a) selecting the subject for a therapy with a platinum-based agent if (i) the tumor sample has a wild-type BRCA1/2 gene mutational status and an immune signature above the threshold level, or (ii) the tumor has a mutated BRCA1/2 gene mutational status, or (b) selecting the subject for no therapy or a therapy other than a therapy with a platinum-based agent if the tumor sample has a wild-type BRCA1/2 gene mutational status and an immune signature below the threshold level.
5. A method comprising: assaying a tumor sample from a subject for a BRCA1/2 gene mutational status selected from wild-type and mutated; optionally assaying the tumor sample identified as having a wild-type BRCA1/2 gene mutational status for an immune signature, wherein the immune signature is above or below a threshold level; designating the subject as a good responder or a poor responder to a therapy with a platinum-based agent based on the BRCA1/2 gene mutational status of the tumor sample.
6. The method of paragraph5, wherein the subject is designated as a good responder if the tumor sample has a mutated BRCA1/2 gene mutational status.
7. The method of paragraph5, wherein the subject is designated as a good responder if the tumor sample has a wild-type BRCA1/2 gene mutational status and the immune signature is above the threshold level.
8. The method of paragraph5, wherein the subject is designated as a poor responder if the tumor sample has a wild-type BRCA1/2 gene mutational status and the immune signature is below the threshold level.
9. The method of any one of claims 5-8 further comprising selecting the subject for a therapy with a platinum-based agent if the subject is a good responder.
10. The method of any one of the preceding claims, wherein the immune signature is a M1 macrophage immune signature generated using an algorithm capable of estimating abundances of member cell types in a mixed cell population using gene expression data, optionally wherein the algorithm is CIBERSORT.
11. The method of any one of the preceding claims, wherein the immune signature is an immune cell-specific enrichment score generated using an algorithm capable of calculating separate enrichment scores for each pairing of a sample and gene set, wherein each enrichment score represents the degree to which genes in a particular gene set are coordinately up-regulated or down-regulated within a sample, optionally wherein the algorithm is single sample Gene Set Enrichment Analysis (ssGSEA).
12. The method of any one of the preceding claims, wherein the immune signature is an immunomodulatory subtype assignment based on the TNBCtype classification system.
13. The method of any one of the preceding claims, wherein the immune signature is based on the measurement of one or more immune gene set.
14. The method of any one of the preceding claims, wherein the tumor sample is from a subject with breast cancer, optionally triple negative breast cancer (TNBC).
15. The method of any one of the preceding claims, wherein the tumor sample is from a subject with ovarian carcinoma (OV).
16. The method of any one of the preceding claims, wherein the platinum-based agent is selected from the group consisting of oxaliplatin, cisplatin, carboplatin, nedaplatin, picoplatin, phenanthriplatin, triplatin, spiroplatin, satraplatin, iproplatin, and satraplatin.
17. The method of any one of the preceding claims, wherein the therapy is a combination therapy that comprises at least one agent in addition to the platinum-based agent.
18. The method of 17, wherein the combination therapy comprises a taxane.
19. The method of 18, wherein the taxane is selected from the group consisting of paclitaxel, docetaxel, and cabazitaxel.
20. The method of paragraph17, wherein the combination therapy comprises cisplatin or carboplatin.
21. The method of paragraph20, wherein the combination therapy comprises docetaxel.

### EXAMPLES

### Example 1: BRCA1 Mutation and Methylation Resulted in the Same TD Phenotype but Associated with Different Therapeutic Response in TNBC

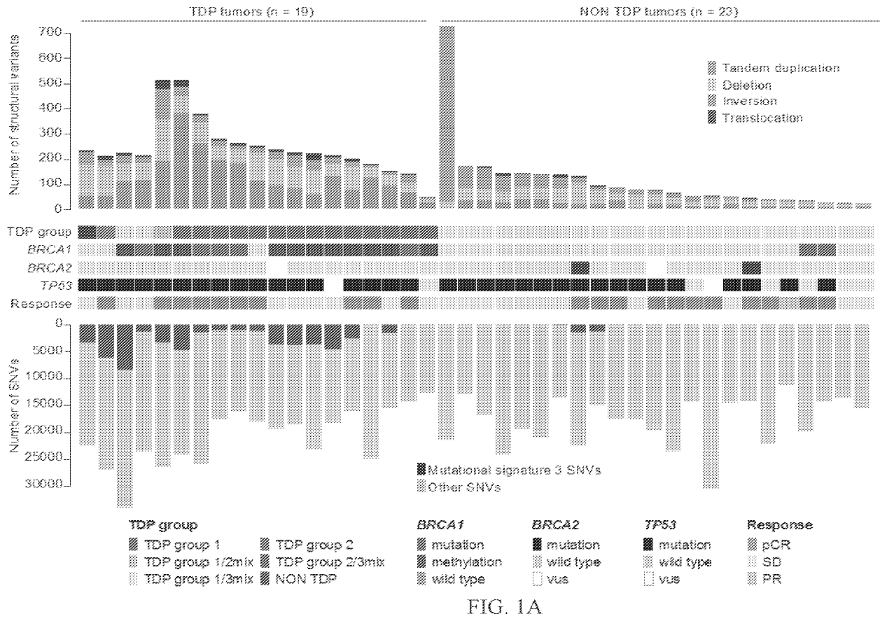

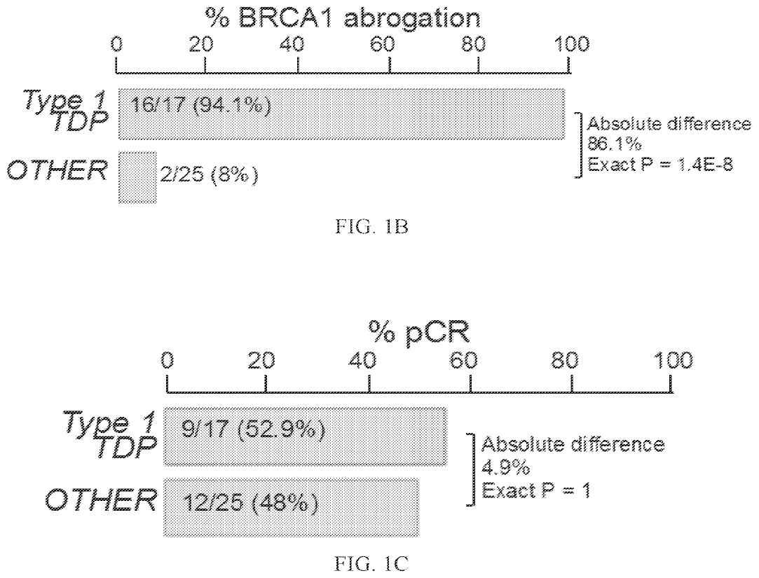

To test the hypothesis that type 1 TDP status may be predictive of optimal response to platinum-based therapy, TDP status was assessed across a cohort of 42 TNBC patients undergoing neoadjuvant carboplatin and NAB-paclitaxel. All tumor specimens were collected as treatment-naïve biopsies before starting the neoadjuvant treatment. 45% of the tumors classified as TDP with (19/42, FIG. 1A). Consistent with a previous observation, 17 of the 19 TDP tumors were classified as type 1 TDPs characterized by short span TDs and were strongly associated with BRCA1 mutation or promoter methylation (16/17, 94.1%, vs. 2/25, 8% of the other tumor types (i.e. TDP group 1 and non TDP), P=1.4E-8, FIG. 1B). However, there was no association between TDP status and pCR (FIG. 1C). The rates of pCR were higher among BRCA1 mutant patients (6/7, 85.7%) compared to either BRCA1 wild type (11/24, 45.8%) or BRCA1 methylated patients (4/11, 36.4%), although the difference did not reach statistical significance (FIG. 1D). Both patients with mutant BRCA2 achieved pCR (FIG. 1A). Moreover, breast tumors from BRCA1/2 germline mutation patients have been previously shown to be sensitive to platinum-based chemotherapeutic regimens. Thus, tumors harboring BRCA1 and BRCA2 disruptive mutations were grouped together. When analyzed in this manner, patients with BRCA1/2 mutations (but not BRCA1 methylation) were more likely to achieve pCR than those with wild type or methylated BRCA1/2 (88.9% vs. 38.7%, P=0.02, FIG. 1E). To exclude that the lower pCR rates associated with BRCA1 methylation could have been due to ineffective BRCA1 abrogation, the expression levels of BRCA1 mutant vs. BRCA1 methylated tumors as assessed by RNA sequencing were compared. It was found that BRCA1 expression levels were significantly lower in BRCA1 methylated tumors compared to either BRCA1 mutant or BRCA1 wild type tumors (FIG. 1F). Thus, BRCA1 methylation reduced BRCA1 transcript levels to deficient levels. In addition, the number and span size distribution profiles between BRCA1 mutant relative to BRCA1 methylated tumors were identical (FIGS. 1G-1H), indicating equivalent loss of BRCA1 activity across the two modes of functional abrogation. Taken together, these results suggested that the BRCA1 effect on genomic instability in the form of TDP induction was separable to its effect on therapeutic response. In addition, it was found that despite BRCA1 mutation or promoter methylation resulting in similar effects on BRCA1 transcriptional dosage, the trend towards higher pCR was restricted to patients carrying tumors with genomic mutations in BRCA1 rather than expression changes through promoter methylation, and that the TDP status alone was not a determinant of chemotherapeutic response.

### Example 2: BRCA1/2 Gene Mutations were More Strongly Predictive of Platinum-Based Therapy Response than TDP Status Across Independent Cohorts of Ovarian Carcinoma

Previously, it was determined that BRCA1 deficiency exhibited the identical effect on inducing the TDP in both TNBC and ovarian cancer (OvCa). If the primary genomic biology of TDP formation were the driver for this chemotherapeutic response, then it should hold across different disease types with the same genomic characteristics. Given that platinum-based chemotherapy is the preferred treatment for OvCa, and given that BRCA2 mutations are more common in OvCa than in TNBC, the hypothesis that BRCA1 or BRCA2 mutational status and not TDP status was predictive of patient response to platinum-based chemotherapy was studied.

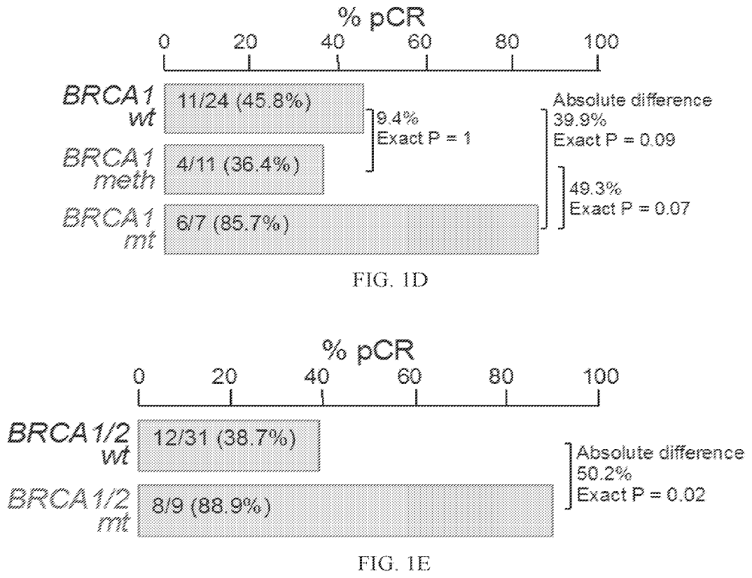

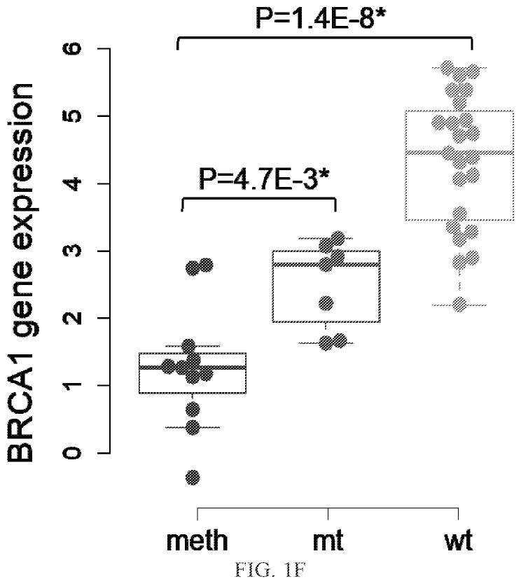

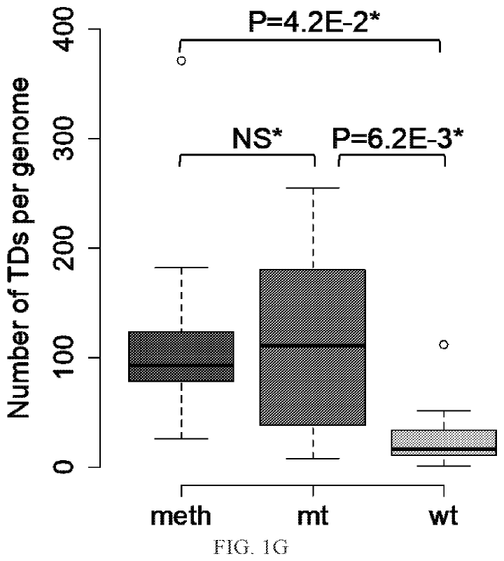

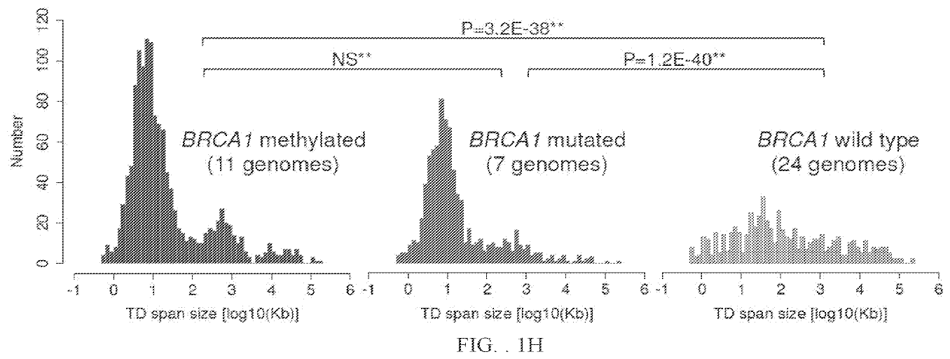

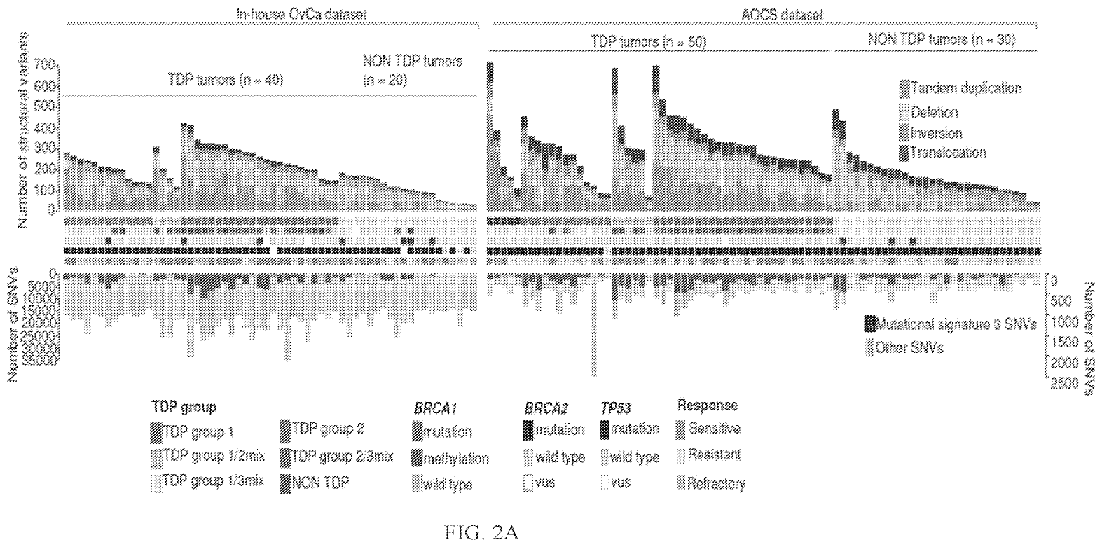

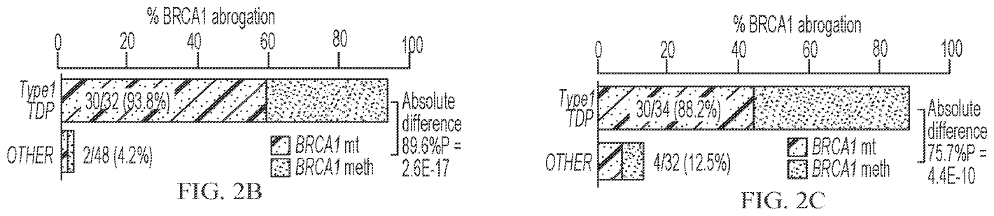

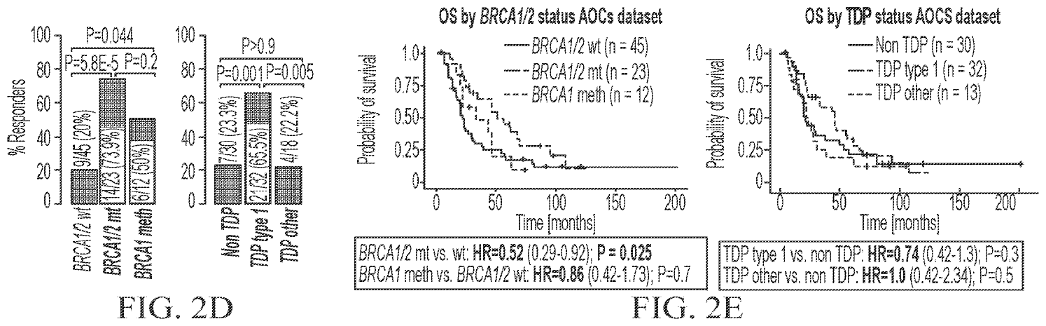

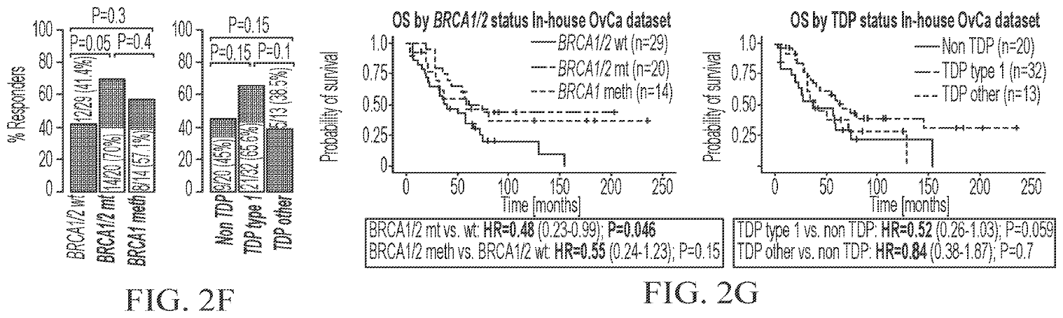

In the first instance, publicly available data was reviewed for ovarian cancer cohorts with complete genome sequence information and response assessment. To this end, the only dataset with detailed response information along with whole genome sequencing and a high number of cases (N=80 primary tumors) was from the Australian Ovarian Cancer Study (AOCS, FIG. 2A). In this dataset, it was observed once again that there was a strong association between both BRCA1 mutation and methylation and TDP type 1 (FIG. 2B). Similar to previous analyses in TNBC, a statistically significant higher rate of platinum sensitivity in patients with BRCA1/2 mutant tumors was observed, compared to those carrying BRCA1/2 wild type tumors, while patients with BRCA1 methylated ovarian tumors showed an intermediate trend of chemosensitivity, as measured by clinical response rate: 73.9% in BRCA1/2 mutant tumors vs. 50% of BRCA1 methylated and 20% of BRCA1/2 wt tumors were registered as “sensitive” to their chemotherapeutic regimen (FIG. 2D). Survival analysis also showed a significant difference between patients classified by their BRCA1/2 status, but not by their TDP status, with BRCA1/2 mutant patients (but not BRCA1 methylated patients) doing better than BRCA1 wild type patients, with an hazard ratio of 0.52 (FIG. 2E).

Given the positive signal from the publicly available data, a more in-depth analysis was pursued in an independent ovarian cancer dataset. To this end, a new cohort of 68 primary ovarian carcinoma (OvCa) patients with detailed response data to therapy with a combination of carboplatin and a taxane-based chemotherapeutic agent was sequenced (in-house OvCa dataset, FIG. 2A).

In this OvCa cohort, the TDP type 1 configuration was again significantly associated with BRCA1-deficiency (30/34 (88.2%) vs. 4/32 (12.5%) for BRCA1 wild type tumors, P=4.4E-10, FIG. 2C), with equal proportions of BRCA1 mutant and methylated tumors defining the BRCA1 deficient group. This confirmed that BRCA1 deficiency was the most important driver for type 1 TDP in this disorder.

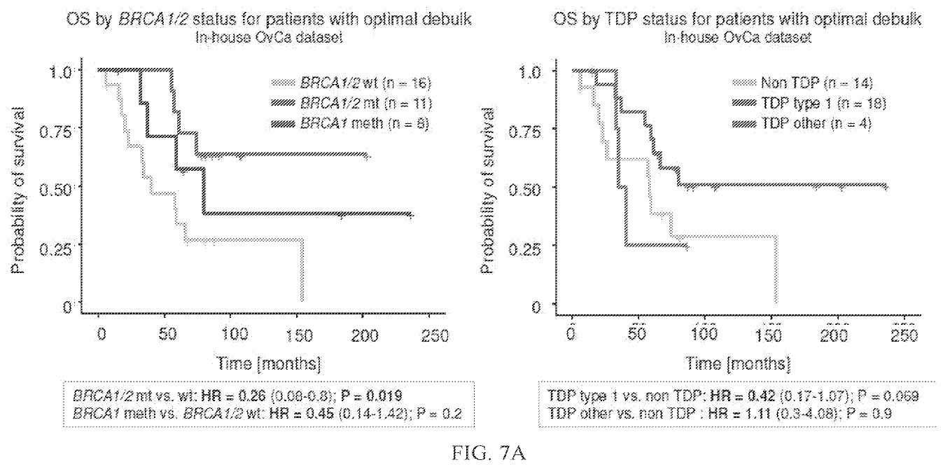

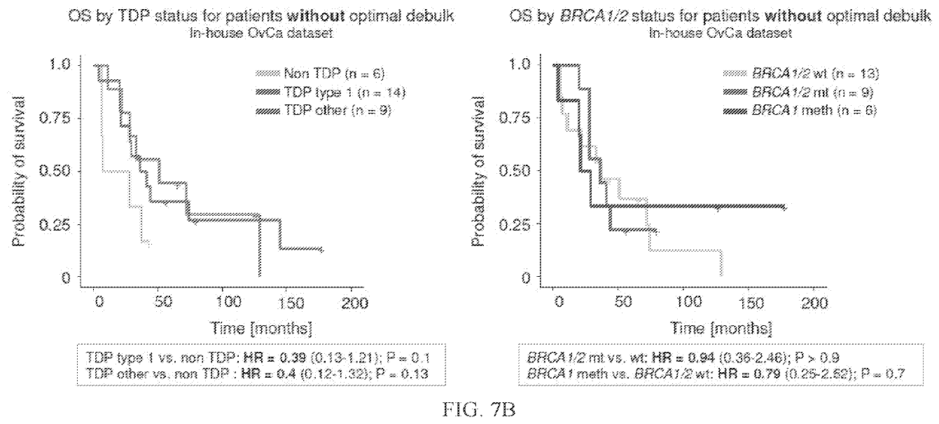

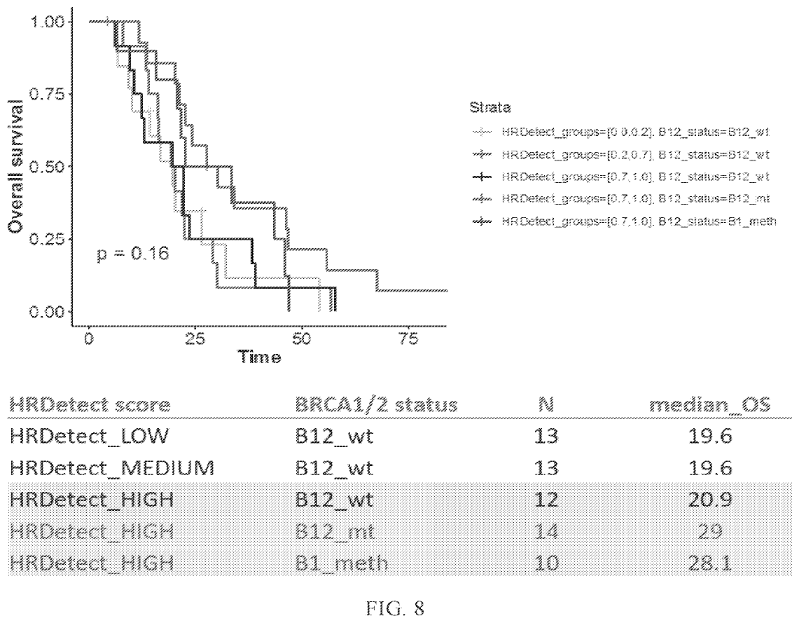

When we examined the clinical response associations with BRCA1/2 status in this new OvCa cohort, again, we found that patients with tumors carrying a BRCA1/2 mutation, but not BRCA1 methylation, significantly associated with better clinical outcomes when compared to patients with BRCA1/2 wild type tumors, both in terms of response rates (70% vs. 41.4%, respectively, P=0.05, FIG. 2F) and of overall survival (HR=0.48, P=0.046, FIG. 2G). Since optimal debulking also appeared to be associated to patient response in a univariate analysis in this dataset, we separated patients who received optimal debulk and those who did not. BRCA1/2 mutational status was highly correlated with better tumor response rates as well as overall survival exclusively in the subgroup of patients who received optimal debulk (FIG. 7A). This indicates again that patients carrying BRCA1/2 mutant tumors are those who specifically benefit from optimal debulking and post-surgical platinum-based chemotherapy. Importantly, neither TDP status, nor BRCA1 methylation status associated with better response, regardless of optimal debulk (FIGS. 7A-7B). Taken together, the analysis of these two OvCa and of one TNBC cohort show that BRCA1/2 mutation status but not BRCA1 promoter methylation, nor TDP status is consistently predictive of platinum response and that these associations are consistent across the two tumor types examined.

### Example 3: BRCA1/2 Abrogation and Response to Carboplatin Response Across 33 PDX Models of Human TNBC

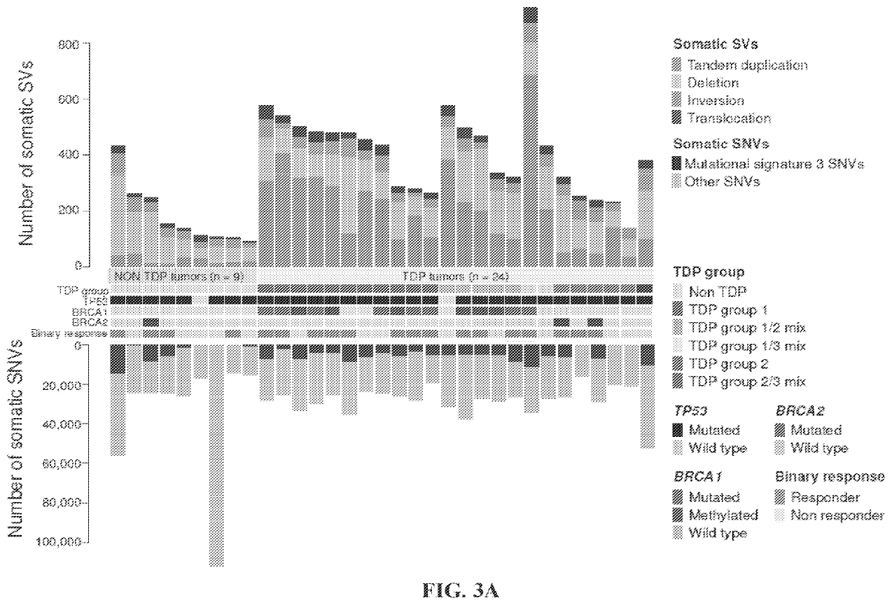

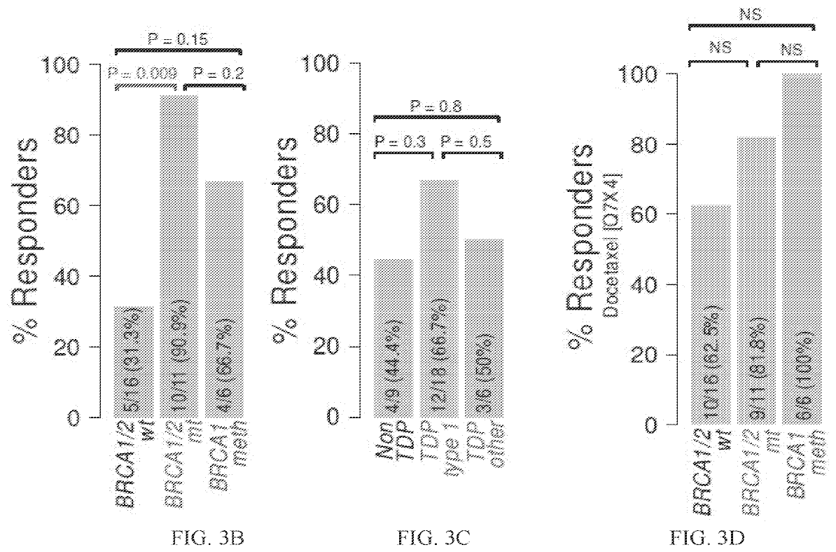

Since this TNBC cohort was treated with a platinum salt in combination with a taxane, it is not possible to attribute the different rates of patient response associated with BRCA1/2 status specifically to the platinum-based treatment. The majority of clinical studies always use combination chemotherapy which makes it difficult to parse out the effects of individual agents. One of the objectives of our study is to ask whether TDP and/or BRCA1/2 status are the key determinants of platinum sensitivity in OvCa and TNBC. To this end, we analyzed a cohort of 33 TNBC patient-derived xenografts (PDXs), treated in vivo with single agent carboplatin (FIG. 3A). comprising 11 PDXs with BRCA1 or BRCA2 deleterious mutations, 6 PDXs with BRCA1 methylated and 16 PDX that were wild type for BRCA1/2. After a 28-day carboplatin regimen, we compared tumor in vivo growth rates between different PDX models classified based on their BRCA1/2 status. We found that only 31.3% of BRCA1/2 wild type tumors treated with carboplatin (i.e. 5/16 tumors) showed a response as defined by a >5% average decrease in tumor growth rate compared to the control arm. By contrast, 90.9% of BRCA1/2 mutant PDXs (10/11, P=0.009) showed a response, an increase of almost three-fold. With 66.7% (4/6) of models showing at least a 5% decrease in their rate of tumor growth, BRCA1 methylated tumors showed once again an intermediate response trend, although this was not statistically significant when compared to the two other PDX groups (FIG. 3B). Importantly, no significant differences in response among the three BRCA1/2 classifications were observed when the same PDXs were treated with docetaxel, as single agent (FIG. 3D), validating our original hypothesis that the BRCA1/2 mutational status exquisitely benefits patients who receive platinum-based chemotherapy. This PDX study therefore offers further validation that the strongest response to platinum-based therapies occurs in tumors with a genetic alteration affecting the BRCA1 or BRCA2 genes, whereas abrogation of BRCA1 via promoter methylation is not statistically associated with better response rates to platinum-based therapies, even though it results in genomic scars virtually indistinguishable from those found in BRCA1 mutant genomes. Finally, though PDX TDP type 1 tumor models show a trend towards better response to carboplatin, this was not statistical significance (FIG. 3C). These data confirm that BRCA1/2 mutational status is a strong predictor of platinum-based response that is better than the genomic phenotype of BRCA1 deficiency, the TDP configuration.

Measures of homologous recombination deficiency (HRD) reflect TDP type 1 status and are not able to distinguish between BRCA1 mutant and methylated tumors, which confounds the prognostic utility of HRD scores for therapeutic response in ovarian and breast cancers.

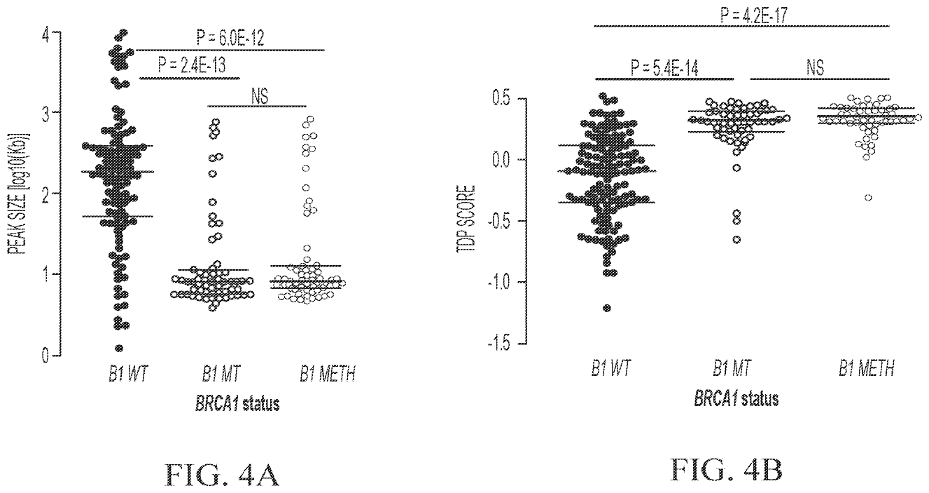

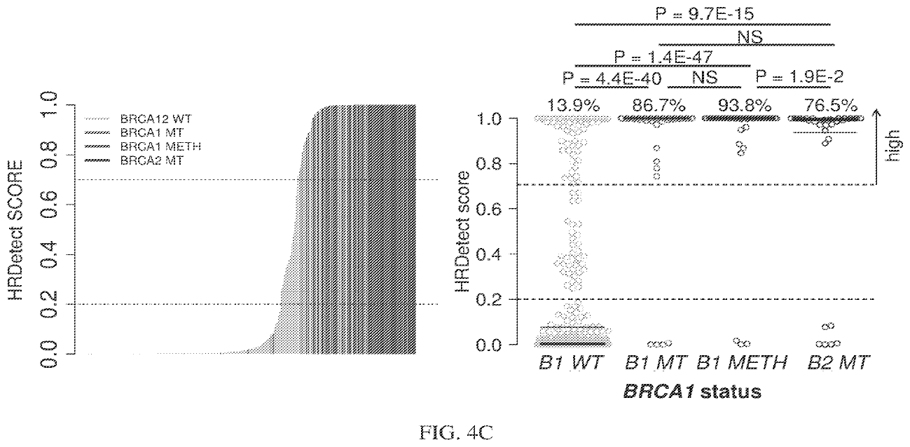

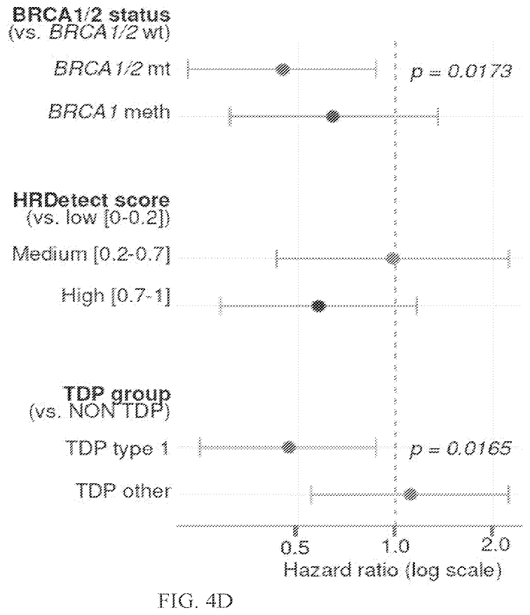

Both BRCA1 mutation and promoter methylation resulted in the same TDP type 1 genomic configuration across all datasets analyzed in this study. However, these two modes of BRCA1 functional inactivation resulted in differential response to therapy. It was therefore hypothesized that mutations in the BRCA1 gene would result in stronger TDP phenotypes compared to BRCA1 promoter methylation. When TDP features across the union of the four examined cancer genome datasets were compared, it was found that while BRCA1 mutated and methylated genomes were both significantly different from BRCA1 wild type genomes, they were remarkably similar in terms of both mode of TD span size distribution, and TDP score, which combines measurements of TD number and genomic scattering (FIGS. 4A-4B). These results emphasized the equivalent functional loss of BRCA1 activity across the two modes of functional abrogation, i.e. mutation and promoter methylation, while suggesting that the BRCA1 effect on genomic instability in the form of TDP induction was separable to its effect on therapeutic response.

It has been postulated that structural genomic instability as measured by HRD scores is a good measure of “BRCAness”, and high HRD scores have been associated with sensitivity to chemotherapy, especially to platinum-based therapies in ovarian cancers. In particular, the HRDetect score has been recently developed to combine and summarize a panel of genomic features associated with BRCA1 and/or BRCA2 deficiency, including the well-established HRD score (e.g., Timms et al. 2014, incorporated by reference herein), single substitution signatures 3 and 8, rearrangement signatures 3 and 5 and the presence of large deletions (>3bps) with microhomology at their junction (e.g., Davies et al. 2017, incorporated by reference herein). To further assess whether BRCA1 mutation and methylation would associate with differences in the type or degree of genomic scars that are linked to BRCAness, beyond the TDP, HRDetect scores between BRCA1 mutant and methylated TNBC and OvCA patients from a meta-cohort of 877 cancer genomes were compared. As expected, there was a highly significant association between both BRCA1 mutation and methylation status with high HRDetect scores (86.7% and 93.8% of each respectively, as compared to 13.9% of BRCA1/2 wild type genomes, FIG. 4C). Importantly, BRCA1 methylated tumors showed identical, if not higher, levels of genomic instability compared to BRCA1 mutant tumors, as measured by HRDetect score (FIG. 4C) as well as each one of the six genomic features that contributed to the score, when these features were assessed individually. BRCA2 mutant tumors also exhibited high HRD scores, confirming that HRDetect predicted for both BRCA1 and BRCA2 deficiencies regardless of the path to functional silencing.

Even though the HRDetect score outperformed the TDP type 1 classifier in capturing BRCA2 deficiencies, these findings suggested that this indicator would suffer the same limitations as the TDP classifier when applied to predict chemo-responsiveness.

We then analyzed therapeutic response in a subset of the AOCS dataset with HRDetect scores and compared overall survival hazard ratios between subsets of patients either based on their BRCA1/2 status or classified using the HRDetect score. The HRDetect classifier was not able to identify the subset of best responders (FIG. 4D) while BRCA1/2 mt status was the best overall predictor in this cohort, being associated with the lowest Hazard Ratio across all comparisons (HR=0.45 vs. BRCA1/2 wt, P=0.0172, FIG. 4D). We then asked whether HRDetect could add treatment response information to those patients with BRCA1/2 wild type tumors to address whether the HRD measure of genomic instability itself would be a marker of chemo-responsiveness. We found that high HRDetect scores did not show any significant improvement in survival compared to patients with tumors with medium or low HRDetect scores (FIG. 4E). Taken altogether, our results show that the determination of the BRCA1 and BRCA2 somatic mutational status is a better predictor of response to platin based therapeutics than the HRD score. Moreover, it is likely that the performance of HRD scores in the clinical setting is highly dependent on the proportion of BRCA1/2 mutant vs BRCA1 methylated vs. BRCA1/2 wild type tumors which would limit the utility of this metric in predicting chemo-responsiveness.

In BRCA1/2 wild type patients, optimal response to neoadjuvant carboplatin/nab-paclitaxel is associated with an enhanced immune signature and specifically a M1 macrophage immune genset expression profile in TNBC and ovarian carcinoma.

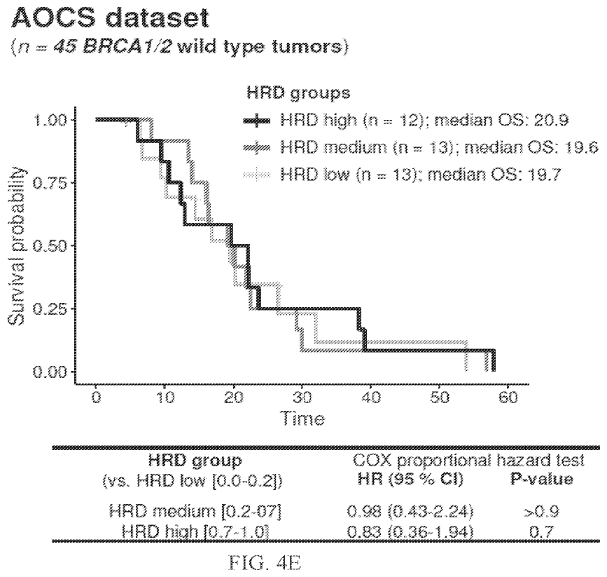

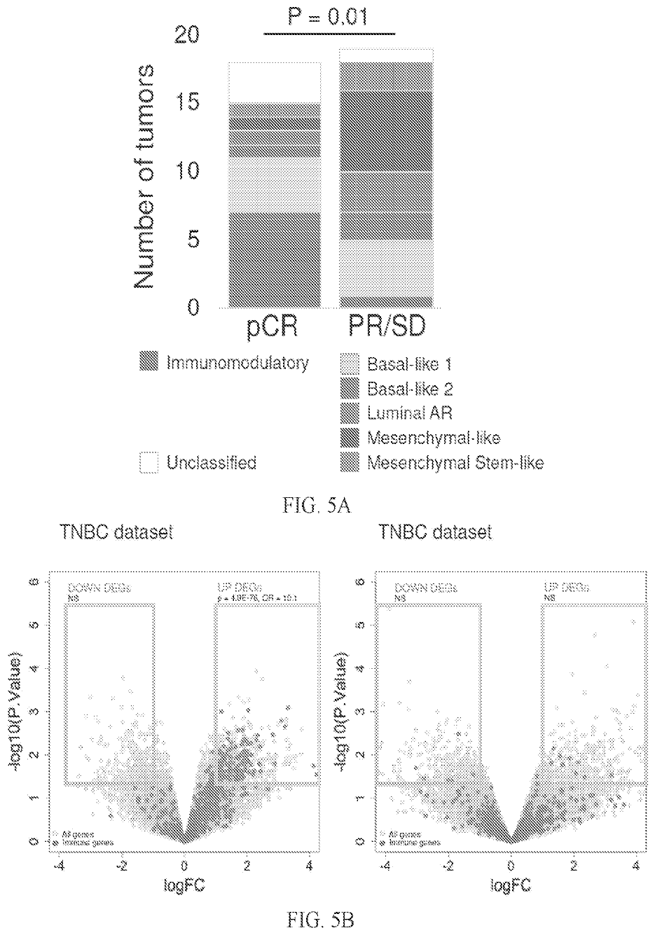

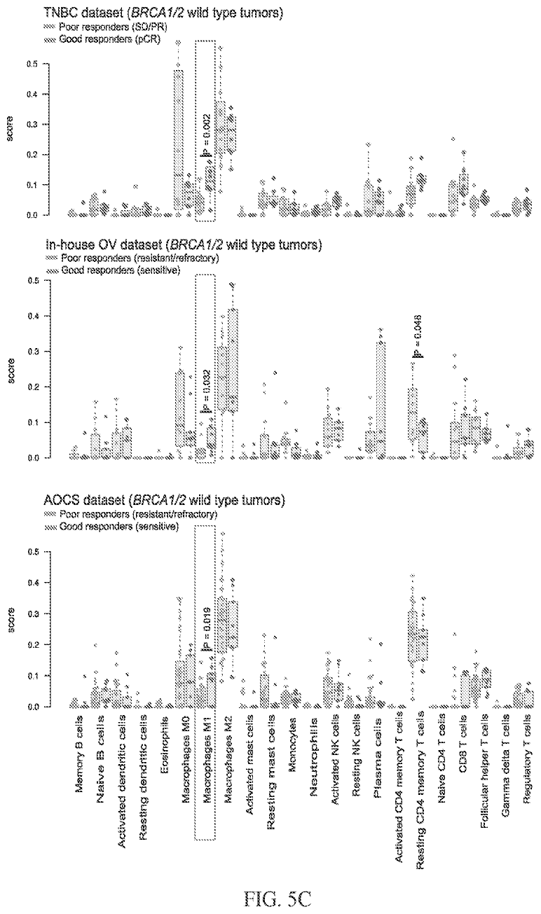

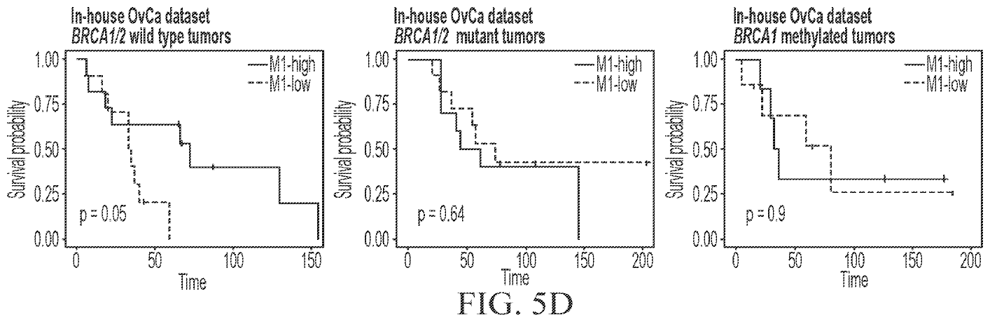

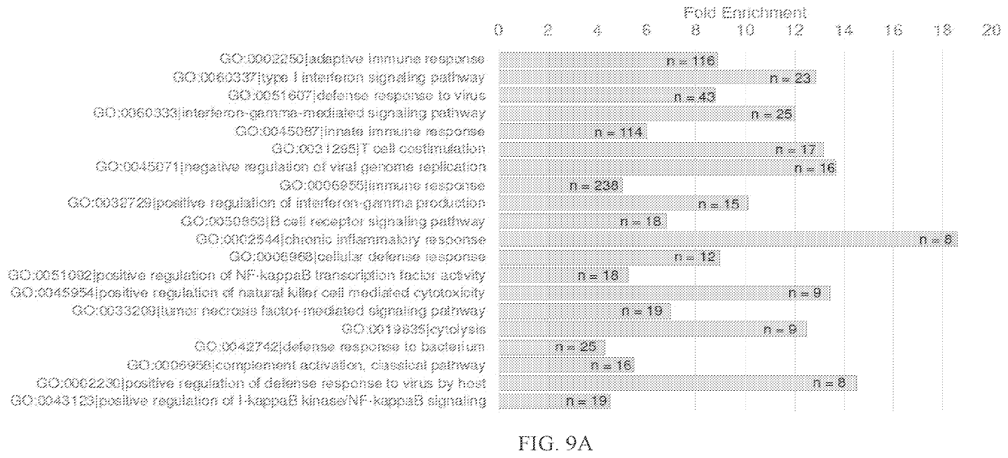

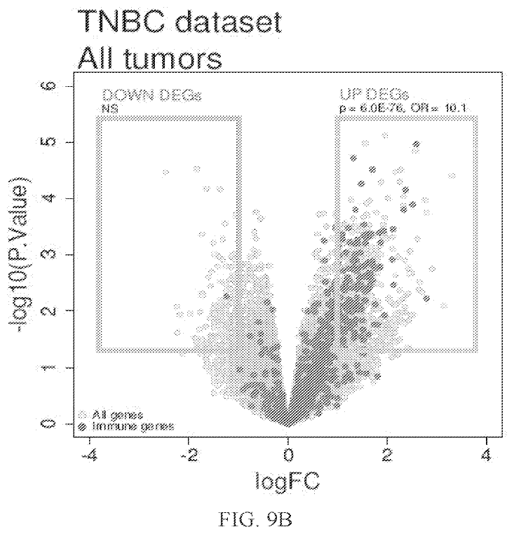

The next step of the study was to identify features that predict patient response in the absence of a BRCA1/2 mutation. RNAseq was used and the gene expression profiles of responders (i.e. pCR) was compared with non-responders (i.e. PR and SD) in the TNBC dataset. Each TNBC was first classified based on the TNBC-type transcriptional classification. Interestingly, when the 6-TNBC subtype classification method was applied, a significant enrichment was observed for the immunomodulatory subtype in the group of BRCA1/2 wild type tumors from patients who achieved pCR (7/15 (46.6%) vs. 1/18 (5.5%), OR=13.6, p=0.01, unclassified tumors are not included, FIG. 4A). immune signatureThis observation prompted the assessment of the type and extent of the immune signature associated with pCR in the TNBC dataset. The entire dataset was first considered, irrespective of BRCA1/2 status, and differentially expressed genes associated with better therapeutic response were identified. As predicted from the TNBC-type analysis, the subset of significantly over-expressed genes was highly enriched for immune response-related pathways, such as adaptive immunity and interferon signaling (FIG. 9A). In fact, a significant proportion of over-expressed genes were annotated as immune genes (p=6.0E-78, FIG. 9B). Remarkably, when the TNBCs were subgrouped based on the BRCA1/2 status, the significant association with overexpression of immune genes was seen only in tumors that were wild type for BRCA1/2 but not in the subset of tumors that harbor BRCA1 methylation (FIG. 5B), suggesting that the detected immune signature may be specifically linked to better response in a BRCA1/2-proficient background. Since this TNBC cohort featured one single non-responder BRCA1/2 mutant tumor, appropriate analysis of differential gene expression in this specific tumor subgroup could not be performed.

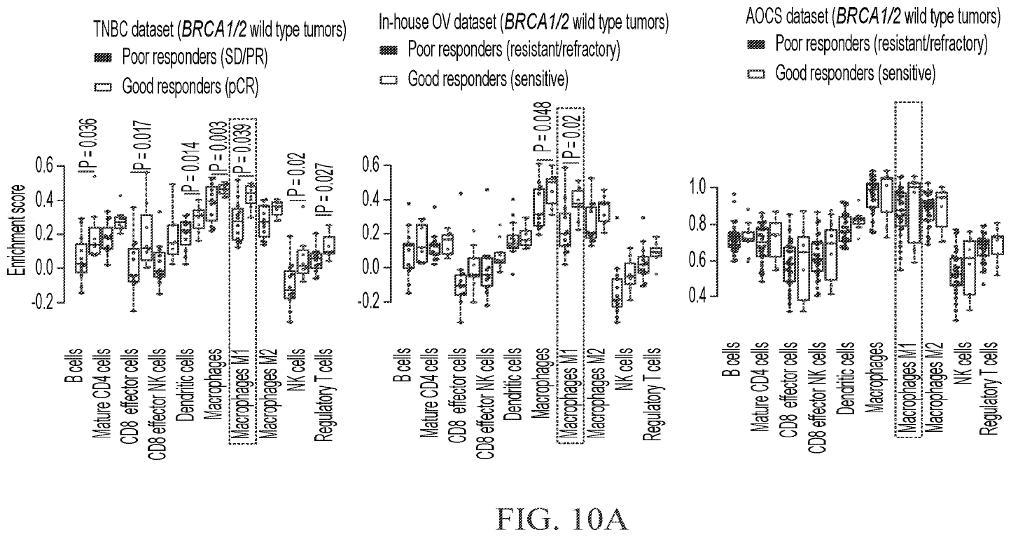

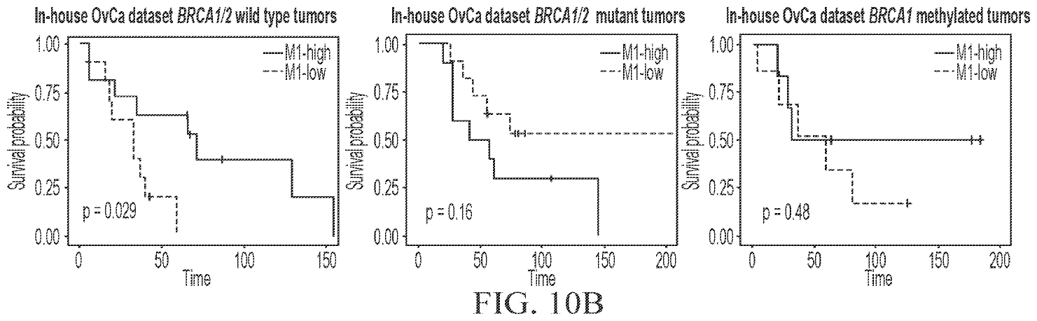

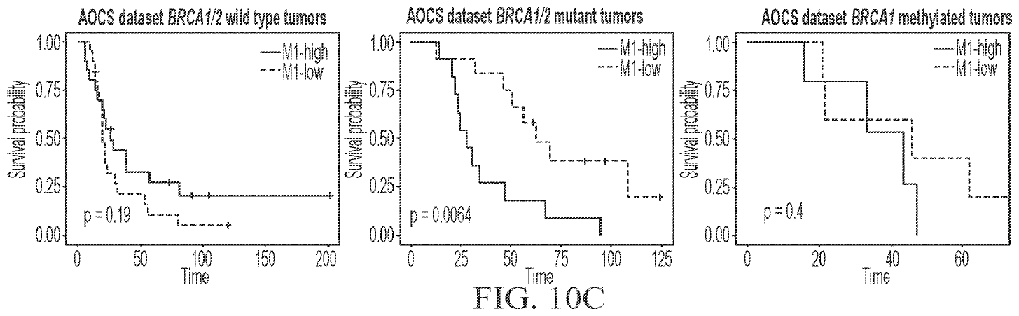

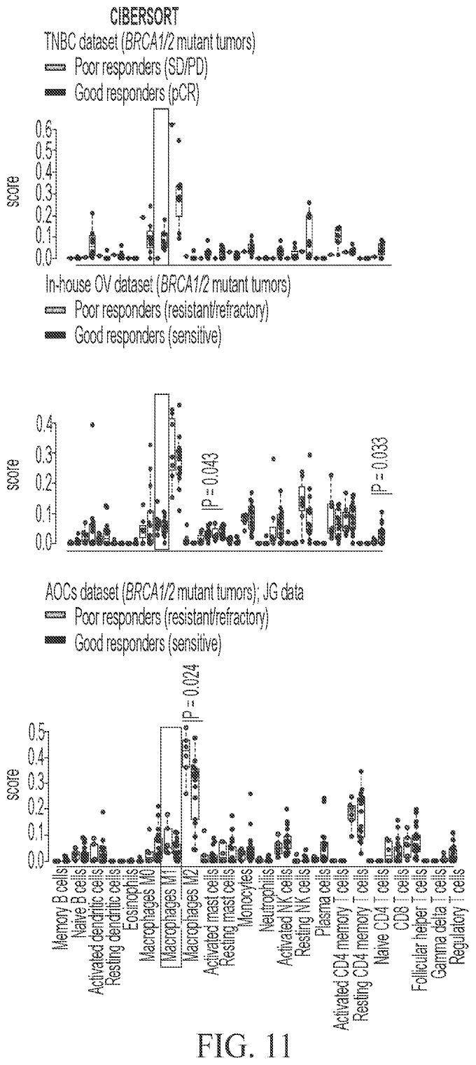

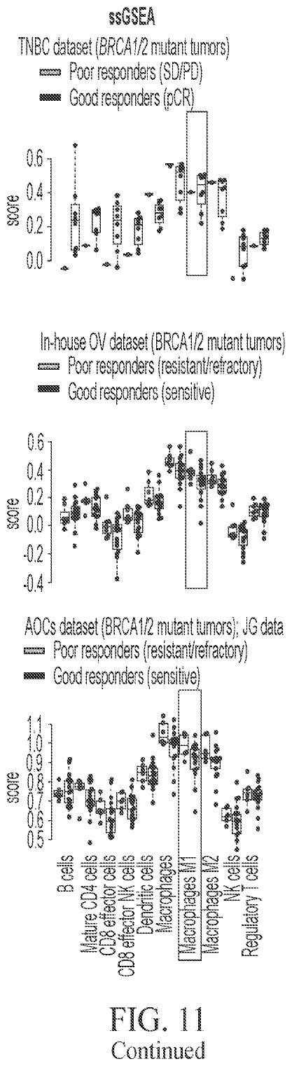

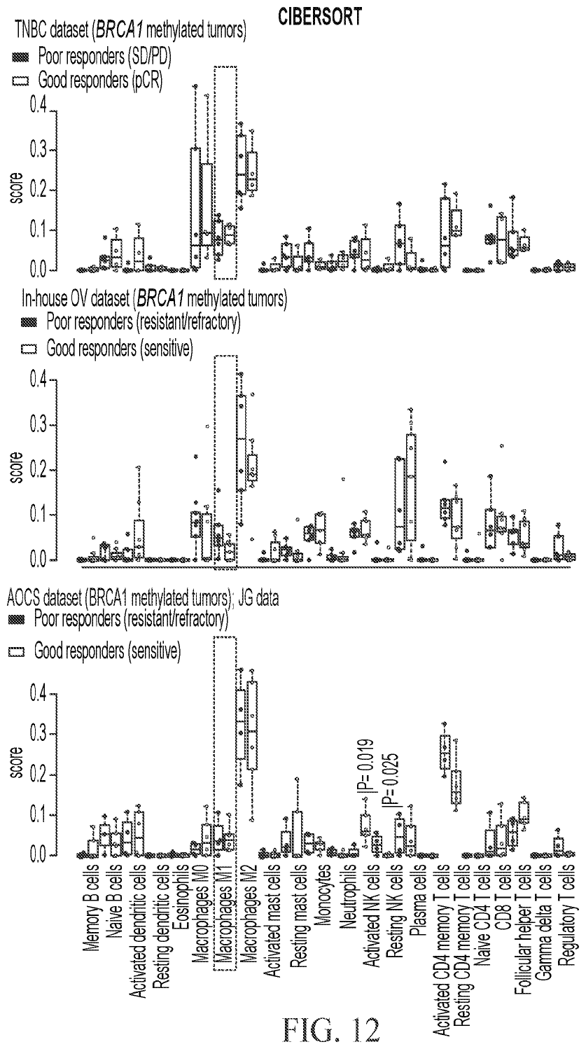

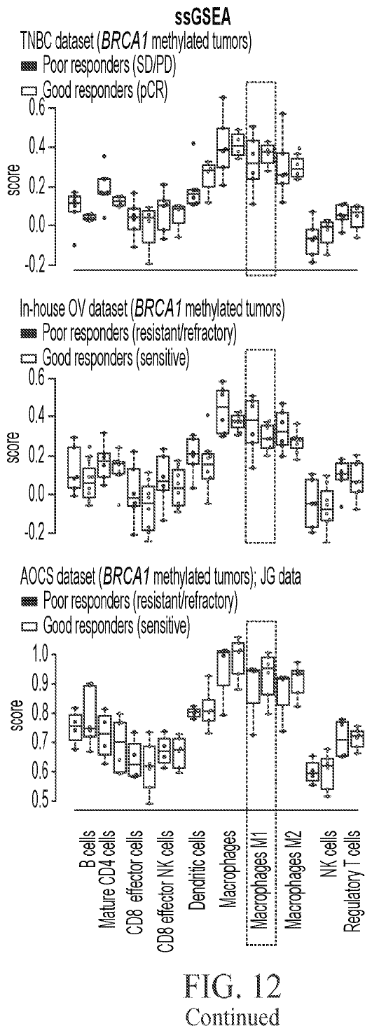

The next step was to see if the observed immune signature could be parsed out into different subsets of infiltrating immune cells. To this end, the CIBERSORT computational approach was applied to the RNA seq data and individual cell fractions from bulk tissue gene expression profiling were quantified. The CIBERSORT-generated scores relative to 22 distinct immune cell components between responder vs. non-responder tumor subgroups in the TNBC dataset were compared, as well as in the two ovarian carcinoma cohorts examined above. Remarkably, the M1 macrophage component was consistently increased in responders vs. non-responders across all three datasets examined. Again, this association was only found when analyzing the subset of BRCA1/2 wild type tumors (FIG. 5C) and did not occur when analyzing the BRCA1/2 mutant and BRCA1 methylated subgroups (FIGS. 11-12). Similar trends were reproduced when a completely alternative methodology of estimating immune cell infiltrates from bulk RNA-seq was applied, namely single sample GSEA analysis, using well defined immune gene signatures as input (FIG. 10A). Even though the comparison did not reach statistical significance in the AOCS BRCA1/2 wild-type dataset, the trend for the M1 gene signature to be enriched in tumors from responsive patients, specifically when on a BRCA1/2 wild type background, but not in any of the other tumor subgroups, was reproduced across all three datasets (FIGS. 11-12). This association was further replicated by analyzing survival curves relative to the two ovarian carcinoma datasets, while splitting patients based on the median M1 enrichment score of their tumors. Again, a high M1 signal associated with significant better overall survival was observed exclusively in the context of BRCA1/2 wild-type genomes (FIGS. 5C-5E, FIGS. 10B-10C). Taken together, these data strongly suggested that the previous association of the immune expression phenotype with better outcome was limited to BRCA1/2 wild type tumors and may be driven by M1 macrophage effects.

### Example 4: A Proposed Decision Tree for Predicting Response to Carbo-Docetaxel Combination Chemotherapy in TNBC and OV

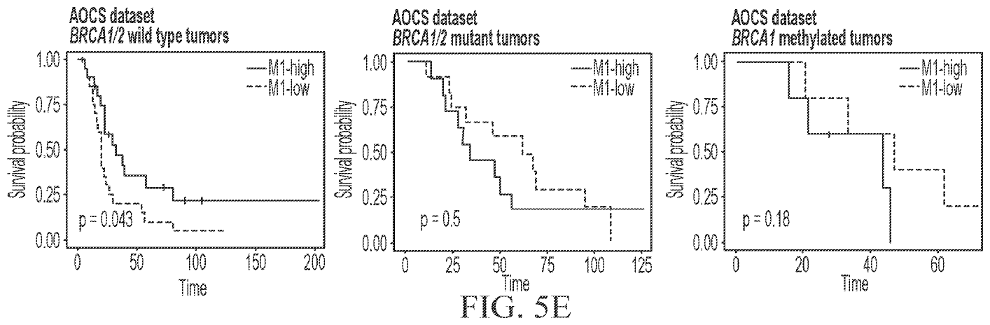

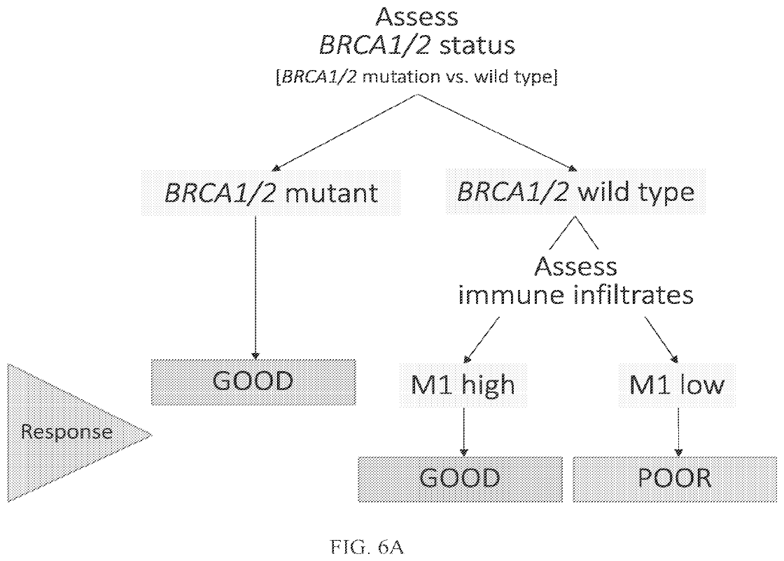

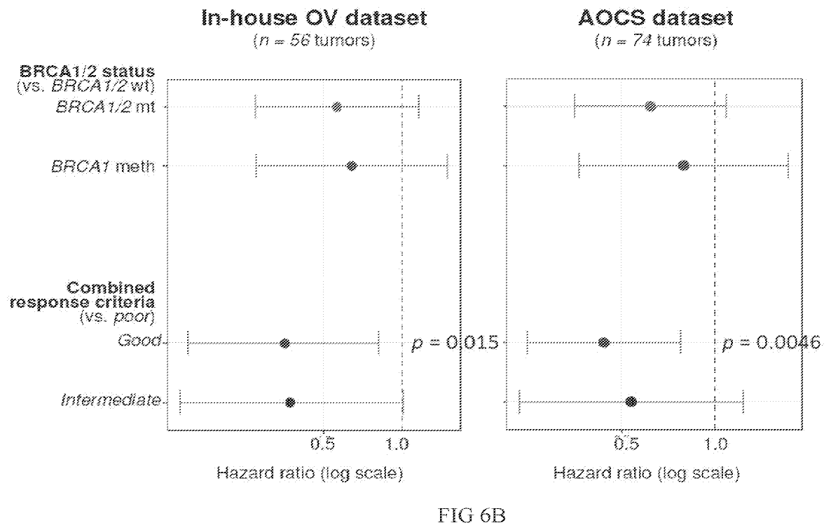

The results strongly suggested that by considering BRCA1/2 mutational status and the expression of an immune signature in BRCA1/2 wild-type tumors, better predictions for chemo-responsiveness can be made than by using BRCA1/2 mutational status alone or HRD scores in TNBC and ovarian cancers. To test this, a response state was developed based on the presence of BRCA1/2 pathogenic mutations and, in the BRCA1/2 wild type tumor subset, on the strength of the M1 macrophage immune signature. Based on this decision tree, each tumor was given an assessment of good vs. poor vs. intermediate response, as indicated in the schematic in FIG. 6A. Briefly, patients with tumors that were mutant for either BRCA1 or BRCA2 were predicted to be good responders. Patients with BRCA1/2 wild type tumors were expected to be good responders only when the M1 macrophage signal was above the median level for that patient cohort, they were otherwise predicted to have a poor response. Patients with BRCA1 methylated tumors were considered to have an intermediate response. The same cohorts were also stratified based on BRCA1/2 status alone. All modes of patient stratification were then compared to the actual clinical outcomes, using both log rank and COX proportional hazard ratio test statistics, to assess and quantify their predictive value. In each of the cohorts examined, the response outcomes predicted by the proposed decision tree outperformed the predictive power of BRCA1/2 status alone, as indicated by lower hazard ratios and p-values (FIG. 6B and FIGS. 13A-13B). This finding further supported the hypothesis that BRCAness status, and consequently HRD, are not good predictors of chemotherapeutic response per se, and that additional patient stratification taking into account how the BRCAness status is achieved in a cancer genome (i.e. mutation vs methylation) is required to for a better outcome prediction. It also highlighted the relevance of BRCA1/2-based patient classification prior to assessing the potential role of immune infiltrates in guiding tumor responsiveness. Taken together, these analyses offered a novel and optimized protocol to predict response to carbo-docetaxel combination chemotherapy in TNBC and OV.

This present disclosure confirmed that reduction of BRCA1 activity via either mutation or methylation robustly associates with type 1 TDPs in TNBC. However, TDP status did not predict good response, suggesting the separation of BRCA effects on genomic instability and platinum sensitivity. This indicated that genomic signature assessments, such as TDP and HRD, may not be sufficient in predicting pathologic complete response (pCR) in TNBC. Importantly, BRCA1/2 mutated TNBC patients were more likely to experience pCR (8/9) compared with patients with either BRCA1 methylation (4/11) or wild type BRCA1/2 (8/21). The exact genetic underpinnings of response in non-BRCA patients are currently under investigation.

### Methods

### TNBC Cohort

Forty-two (42) patients with TNBC were enrolled in a phase II study of neoadjuvant carboplatin/nab-paclitaxel at the City of Hope National Medical Center (NCT01525966). Pathological complete response (pCR) was achieved in 50% of pts (21/42). Whole-genome sequencing (WGS) was performed using standard Illumina protocols. Structural variants were called using Crest, Delly and BreakDancer, and high confidence breakpoints were selected when called by at least two tools and by requiring split-read support. TDP status was ascertained as recently described (2). BRCA1 methylation was determined by methylation-specific PCR. Vanderbilt TNBC subtypes were determined using the TNBCtype-4 tool.

### Ovarian Carcinoma Cohorts

University of Washington (OvCa) dataset. A total of 68 serous ovarian carcinomas were selected from the Gynecologic Oncology Tissue Bank established by Prof. Elizabeth M. Swisher at the University of Washington, Seattle, WA, to represent extreme outcomes of patient survival but independently of any other genetic or clinical features. Australian Ovarian Cancer Study (AOCS). This dataset comprises 80 primary serous ovarian carcinomas that are part of the Australian ovarian cancer study (AOCS). Their BRCA1/2 status had been previously published (Patch et al., 2015). Structural variant calls were downloaded from the COSMIC data portal (data freeze version v78). Clinical data and RNAseq-based gene expression were downloaded from the ICGC data Portal (dcc.icgc.org/) in August 2020.

### Patient-Derived Xenograft (PDX)

Tumor volumes in the control and treatment arm were assessed over a period of 28 days, with saline (control arm) and carboplatin (treatment arm) dosing on days 1, 7, 14 and 21. The rate of tumor growth for each animal in the study was computed by fitting a linear curve to the log-transformed tumor volumes. Finally, the percentage difference in tumor growth rate for each animal was calculated by comparing its tumor growth rate to the average growth rate of all the tumors in the control arm.

### Example 5. Loss of BRCA1 Promoter Methylation and Induction of BRCA1 Expression in PDX Models of TNBC after Platinum-Based Chemotherapy

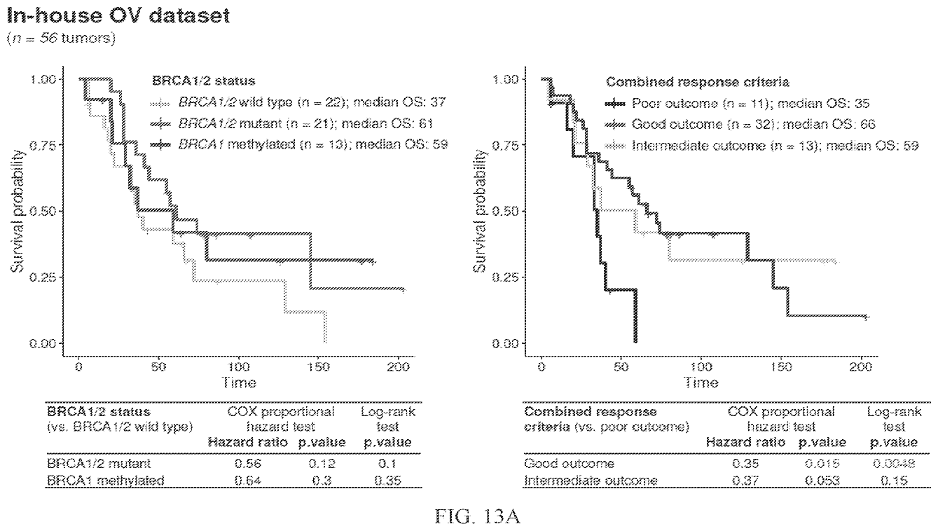

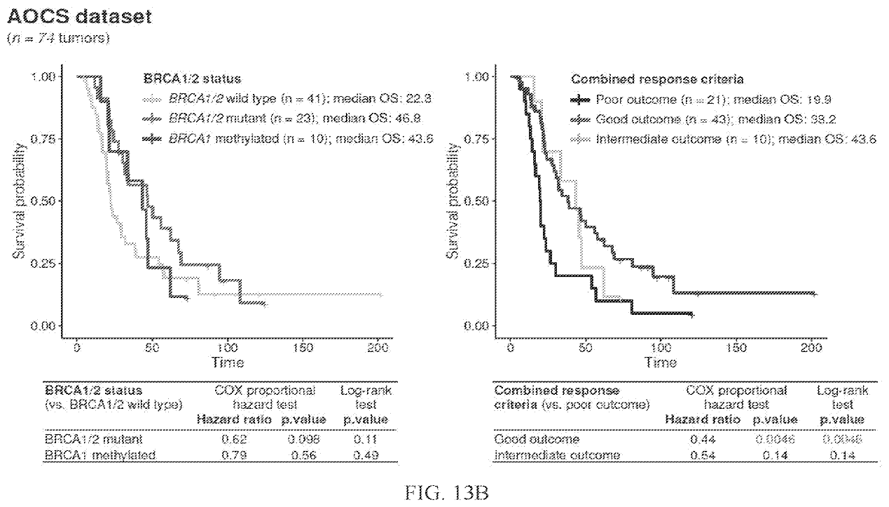

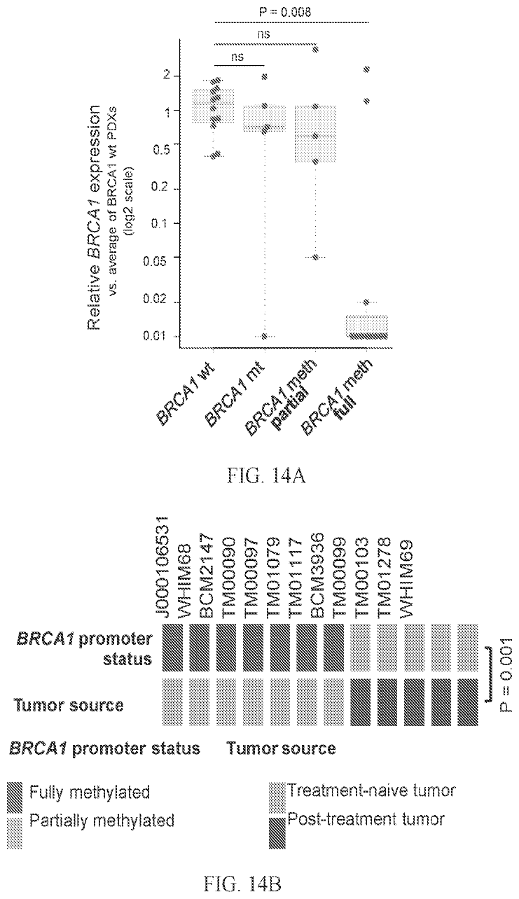

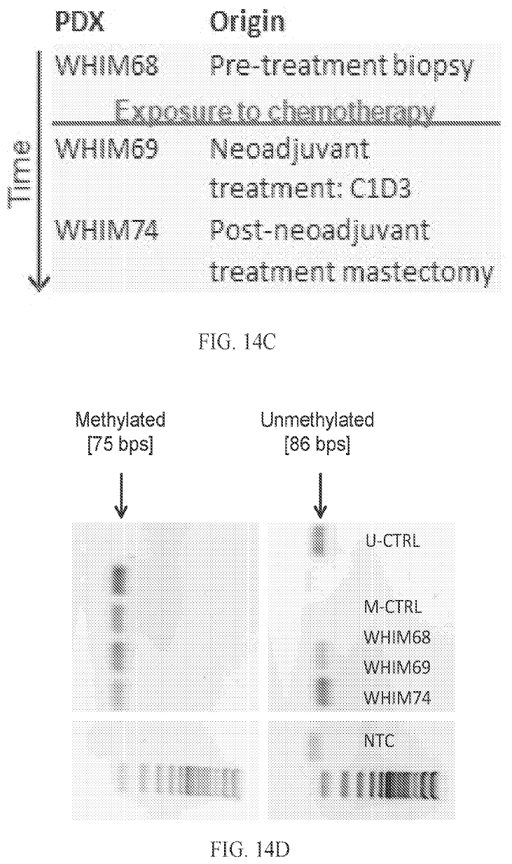

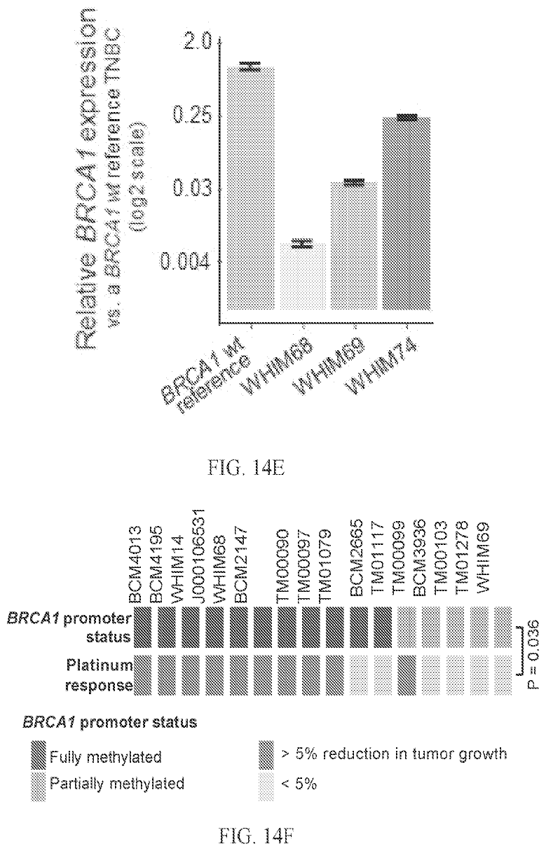

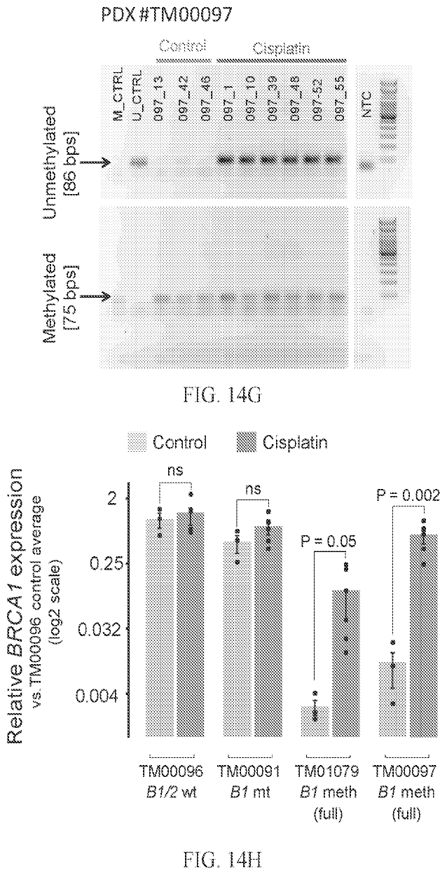

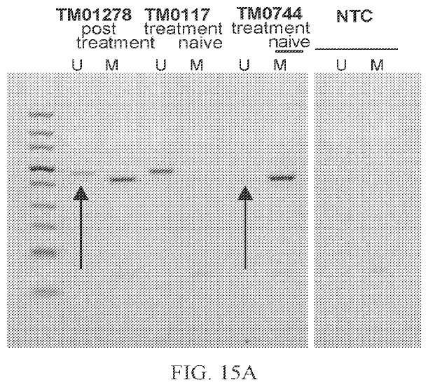

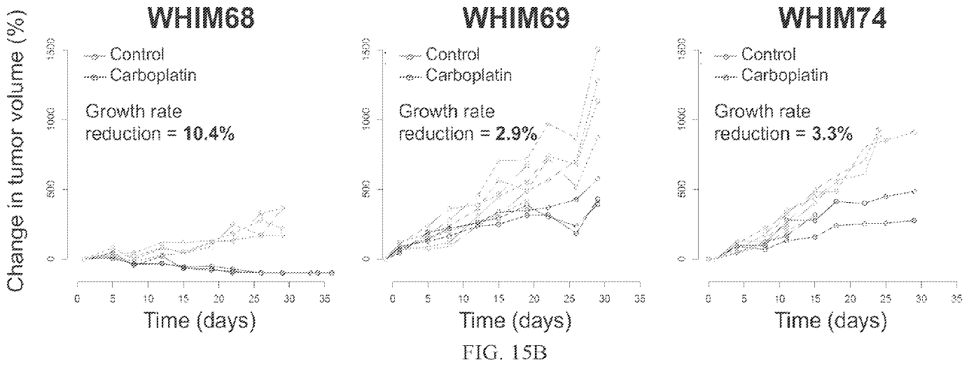

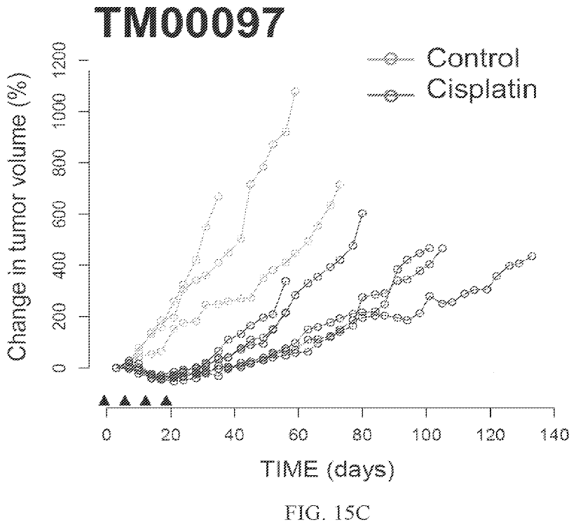

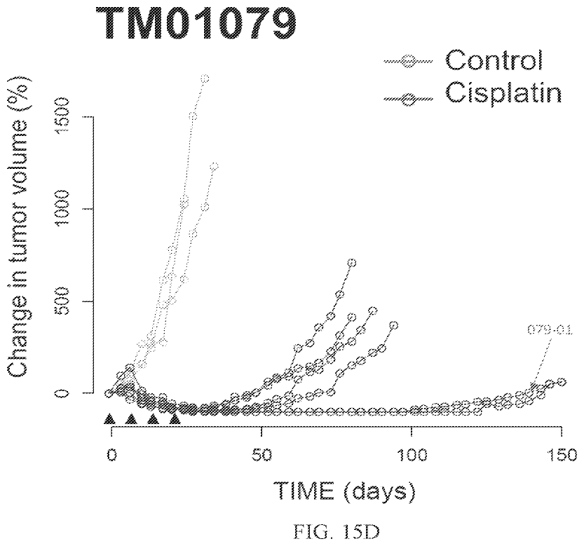

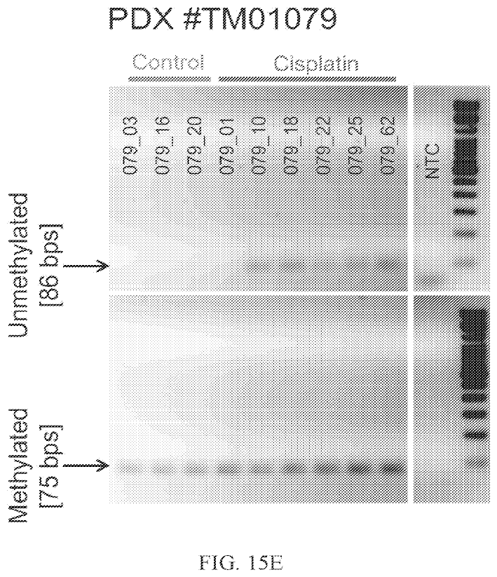

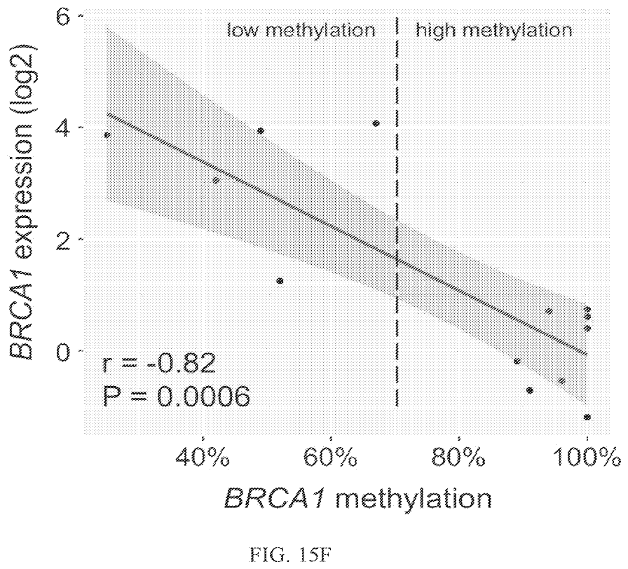

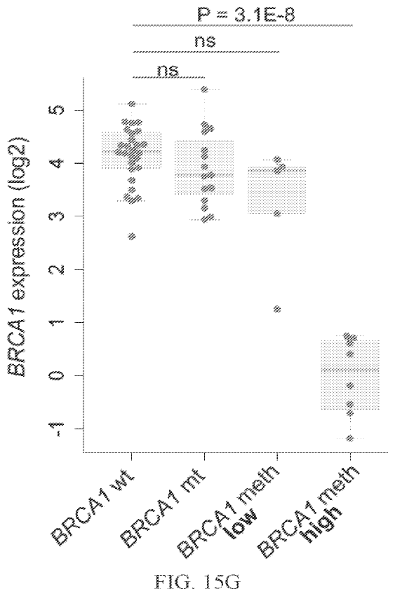

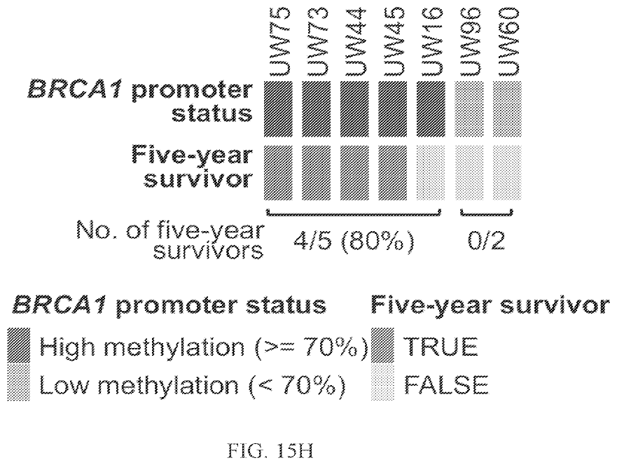

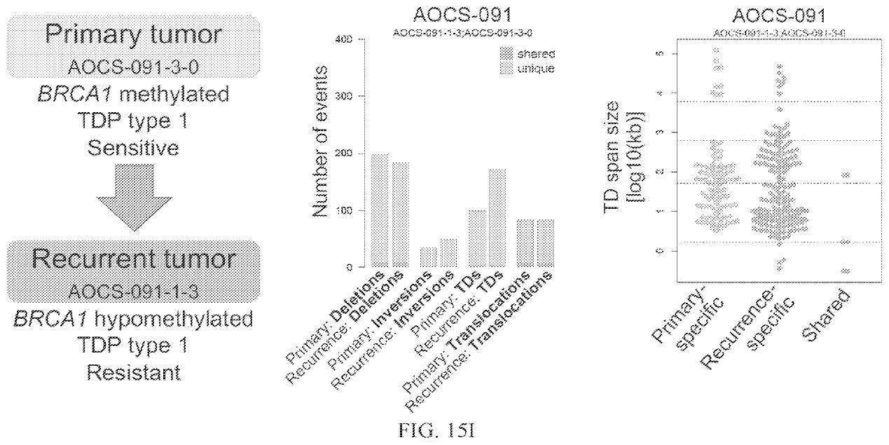

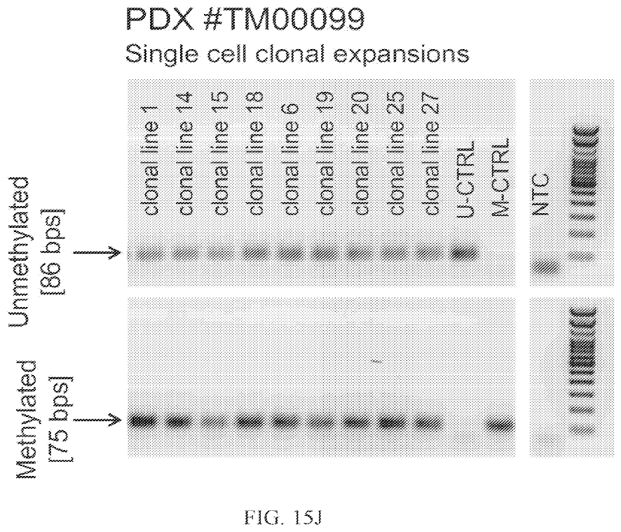

BRCA1 methylated tumors did not show the same sensitivity to platinum-based chemotherapies as BRCA1 mutated tumors, which was puzzling given the functional equivalence between the two states in terms of downstream genomic effects. To address this question, a total of 12 PDX models of BRCA1 methylated TNBC were first examined, combining the subset of BRCA1 methylated tumors from the PDX cohort described above and additional BRCA1 methylated tumors available from The Jackson Laboratory PDX Resource, all of which shared the TDP Type 1 configuration and whose patient donors' clinical histories prior to the model establishment were available. Two modes of BRCA1 promoter methylation were observed, as assessed by Methylation-Specific PCR (MSP): (a) full methylation (i.e., no signal for the unmethylated PCR product) and (b) partial methylation (i.e., two signals corresponding to both the methylated and the unmethylated PCR products, FIG. 15A). While MSP is not a quantitative assay per se, the absence of normal human DNA contamination in the PDX system justifies the assumption that a full methylation profile corresponds to homozygous BRCA1 methylation (i.e., all copies of the BRCA1 promoter are methylated), whereas partial methylation indicates either a heterozygous profile or subclonal heterogeneity. It was then asked whether these promoter methylation configurations had an impact on BRCA1 gene expression. It was found that the full promoter methylation status associated with a significant decrease in BRCA1 expression levels when compared to BRCA1 wild type TNBC PDXs, while partial promoter methylation showed only a marginal reduction: median BRCA1 expression values were reduced to 1% and 59% of the wild type BRCA1 levels for fully and partially methylated PDXs, respectively, FIG. 14A. Intriguingly, it was also observed that while all of the PDX models established from treatment-naïve patient tumors showed full methylation of the BRCA1 promoter (n=8/8), those which had been established from patient tumors after the exposure to chemotherapeutic regimens (i.e., post-treatment) consistently showed only partial methylation (n=5/5, P=0.001, FIG. 14B).

The relationship between BRCA1 methylation status and treatment exposure was further investigated using three PDX models, WHIM68, WHIM69 and WHIM74, established from subsequent biopsies and surgical specimens from the same TNBC patient donor: WHIM68 was established from a pre-neoadjuvant treatment biopsy and it was therefore treatment-naïve, WHIM69 was derived from a research biopsy taken at day 3 after the first cycle of neoadjuvant treatment with carboplatin and docetaxel; and WHIM74 was derived from a surgical specimen obtained after the completion of the neoadjuvant course (FIG. 14C). To determine whether the chemotherapeutic treatment of an individual patient would result in progressive loss of BRCA1 promoter methylation while augmenting steady state BRCA1 expression, this PDX series was used. The BRCA1 methylation status of the three serial PDXs was first assessed via both MSP and quantitative methylation specific droplet digital PCR (MS-ddPCR) and confirmed that WHIM68 was the only PDX in this series to have a homozygous BRCA1 methylation profile. By contrast, the two remaining PDXs, showed the presence of an unmethylated signal (FIG. 14D). BRCA1 gene expression, as assessed by quantitative PCR, was nearly undetectable in the WHIM68 model, but progressively increased in each successive PDX model (FIG. 14E). This suggests that loss of BRCA1 methylation can occur shortly after a single exposure to chemotherapy, and that the resultant tumors progressively increase BRCA1 expression after successive chemotherapeutic exposures. Importantly, the loss of BRCA1 methylation in this PDX series appeared to be associated with relative resistance to carboplatin in vivo (FIG. 15B). Therefore, the possible association between BRCA1 methylation status and platinum-therapy response was explored by expanding the BRCA1 methylated TNBC PDX cohort to a total of 16 models with vivo response to either cisplatin or carboplatin therapy. It was found that PDXs with full methylation of the BRCA1 promoter had significantly higher response rates compared to those with partial methylation (82% response rate (n=11) vs. 20% response rate (n=5), P=0.0036, and FIG. 14F).

It was then evaluated whether direct treatment with chemotherapy of a PDX can cause loss of BRCA1 methylation, by selecting a single fully methylated TNBC PDX established from a treatment-naïve patient tumor (TM00097) and subjecting it to treatment with four weekly doses of either cisplatin or vehicle control, followed by continuous monitoring of tumor recurrence (FIG. 15C). Interestingly, it was found that while only one of the three tumors in the control arm showed a faint unmethylated signal, all of the six cisplatin recurrences showed the emergence of a strong unmethylated signal, clearly suggesting that chemotherapeutic exposure was the cause of the epigenetic shift at the BRCA1 promoter locus (FIG. 14G). Similar results were obtained with a second treatment-naïve and fully methylated PDX model (TM01079), with all three tumors in the control arm maintaining a fully methylated BRCA1 promoter, but five out of six cisplatin recurrences showing the emergence of the unmethylated signal (FIGS. 15D-15E). Importantly, loss of methylation was accompanied by re-expression of the BRCA1 gene to levels often comparable to those found in partially methylated and BRCA1 mutant tumors (FIG. 14H).

To test whether similar trends would translate to the clinical setting, the BRCA1 methylation status of the UW OvCa cohort was re-assessed via quantitative MS-ddPCR. After correcting for the proportion of neoplastic cellularity, the degree of BRCA1 methylation showed a significant negative correlation with BRCA1 expression levels (r=−0.82, P=0.0006, FIG. 15F). Based on a distinct threshold of BRCA1 methylation set at 70%, 10 out the 14 (71.4%) BRCA1 methylated tumors in the cohort were classified as highly methylated and showed a significant decrease in BRCA1 expression when compared to BRCA1 wild type tumors. On the other hand, all tumors <70% BRCA1 methylation (i.e., low methylation) showed levels of BRCA1 expression comparable to those of BRCA1 wild type tumors (FIG. 15G). The modest number of patients in the high methylation group did not permit a proper assessment of the clinical benefit associated with this profile in terms of prolonged overall survival. However, when patients were stratified based on their BRCA1 methylation profile and optimal debulk, it was found that 4/5 patients with high methylation achieved the five-year survival mark, but neither of the two patients with low methylation did (FIG. 15H). While the numbers are too small to assess statistical significance, this trend, specific for the subset of patients who received optimal debulking, is consistent with the observations in the PDX cohort.

A proposed hypothesis for the loss of BRCA1 methylation following chemotherapeutic treatment and the emergence of resistant tumor recurrences, is the rapid expansion of a non-methylated subclone from a heterogeneous tumor cell population that contained BRCA1 methylated and BRCA1 proficient clones. In a previous AOCS study, a patient initially diagnosed with a BRCA1 methylated, platinum sensitive primary ovarian carcinoma recurred with a tumor that was completely unmethylated and chemo resistant. Based on the unexpected observation of a minimal overlap between the somatic single nucleotide mutations observed in primary and the recurrent tumors, it was speculated that the recurrence may have originated from an original BRCA1 non-methylated (therefore BRCA1 proficient) and platinum resistant subclone, which was expanded during the chemotherapeutic treatment. In previous work, it was found that BRCA1 deficiency is a prerequisite for the generation of a TDP genomic configuration in the resultant tumors and potentially is the earliest inciting event. Through a detailed genomic analysis of structural mutations in this tumor pair, it was found that, despite featuring a predominance of private rearrangements, both the primary and the recurrent cancer genomes classified as TDP type 1 (FIG. 15I), indicating that the recurrent tumor originated from a subclone that, early in its evolutionary history, had experienced significant BRCA1 deficiency in order to generate BRCA1-related genomic scars, most likely from BRCA1 promoter methylation, before eventually losing BRCA1 methylation at a later timepoint. To determine whether the observed BRCA1 partial methylation configuration originated from a non-methylated subclone or because of active loss of methylation (i.e., conversion of one BRCA1 promoter allele from the methylated to the unmethylated state), single cell clonal expansions were established from a TNBC PDX model, TM0099, which is a TDP group 1 tumor with two copies of the BRCA1 gene and a partially methylated BRCA1 promoter. MSP analysis of each of the nine individual clonal lines examined showed both methylated and unmethylated products (FIG. 15J). This suggests that, at least in the TM0099 model, loss of methylation was the result of demethylation of one of the two BRCA1 promoter alleles, and not from the expansion of a non-methylated tumor subclone.

Taken together, these data suggest that BRCA1 deficiency due to promoter methylation can be overridden by demethylation of one allele associated with induction of wild type BRCA1 expression, and that this reversion of the methylated state can occur after only a short course of platinum chemotherapy. Finally, once this partially methylated state is established, the resultant tumor exhibits relative resistance to platinum drugs akin to BRCA1 wild type tumors.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

### REFERENCES

1. Barbie et al., Systematic RNA interference reveals that oncogenic KRAS-driven cancers require TBK1. Nature. 2009 Nov. 5; 462(7269):108-12. doi: 10.1038/nature08460. Epub 2009 Oct. 21.
2. Becht et al., Estimating the population abundance of tissue-infiltrating immune and stromal cell populations using gene expression. Genome Biol. 2016 Oct. 20; 17(1):218. doi: 10.1186/s13059-016-1070-5.
3. Bouwman et al., Molecular pathways: how can BRCA-mutated tumors become resistant to PARP inhibitors? Clin Cancer Res. 2014 Feb. 1; 20(3):540-7. doi: 10.1158/1078-0432.CCR-13-0225. Epub 2013 Nov. 22.
4. Chen et al., TNBCtype: A Subtyping Tool for Triple-Negative Breast Cancer. Cancer Inform. 2012; 11:147-56. doi: 10.4137/CIN.S9983. Epub 2012 Jul. 24.
5. Chen et al., Profiling Tumor Infiltrating Immune Cells with CIBERSORT. Methods Mol Biol. 2018; 1711:243-259. doi: 10.1007/978-1-4939-7493-1_12.
6. Coyne et al., Functional characterization of BRCA1 sequence variants using a yeast small colony phenotype assay. Cancer Biol Ther. 2004 May; 3(5):453-7. doi: 10.4161/cbt.3.5.809. Epub 2004 May 18.
7. Davies et al., HRDetect is a predictor of BRCA1 and BRCA2 deficiency based on mutational-signatures. Nat Med. 2017 April; 23(4):517-525. doi: 10.1038/nm.4292. Epub 2017 Mar. 13.
8. Davoli et al., Tumor aneuploidy correlates with markers of immune evasion and with reduced response to immunotherapy. Science. 2017 Jan. 20; 355(6322):eaaf8399. doi: 10.1126/science.aaf8399.
9. Monteiro et al., Yeast-based assays for detection and characterization of mutations in BRCA1. Breast Disease. 1998; 10:61-70.
10. Morris et al., Genetic analysis of BRCA1 ubiquitin ligase activity and its relationship to breast cancer susceptibility. Hum Mol Genet. 2006 Feb. 15; 15(4):599-606. doi: 10.1093/hmg/ddi476. Epub 2006 Jan. 10.
11. Patch et al., Whole-genome characterization of chemoresistant ovarian cancer. Nature. 2015 May 28; 521(7553):489-94. doi: 10.1038/nature14410.
12. Scully et al., Genetic analysis of BRCA1 function in a defined tumor cell line. Mol Cell. 1999 December; 4(6):1093-9. doi: 10.1016/s1097-2765(00)80238-5.
13. Timms et al., Association of BRCA1/2 defects with genomic scores predictive of DNA damage repair deficiency among breast cancer subtypes. Breast Cancer Res. 2014 Dec. 5; 16(6):475. doi: 10.1186/s13058-014-0475-x.
14. Wu et al., Functional evaluation and cancer risk assessment of BRCA2 unclassified variants. Cancer Res. 2005 Jan. 15; 65(2):417-26.

1. A method comprising:
assaying a tumor sample from a subject for a wild-type BRCA1 and BRCA2 (BRCA1/2) gene mutational status; and
assaying for an immune signature in the tumor sample.

2. The method of claim 1 further comprising selecting the subject for a therapy with a platinum-based agent if the immune signature is above a threshold level.

3. The method of claim 1 further comprising selecting the subject for no therapy or a therapy other than a therapy with a platinum-based agent if the immune signature is below a threshold level.

4. The method of claim 1, wherein the BRCA1/2 gene mutational status is selected from wild-type and mutated, and the method further comprises (a) selecting the subject for a therapy with a platinum-based agent if (i) the tumor sample has a wild-type BRCA1/2 gene mutational status and an immune signature above a threshold level, or (ii) the tumor sample has a mutated BRCA1/2 gene mutational status, or (b) selecting the subject for no therapy or a therapy other than a therapy with a platinum-based agent if the tumor sample has a wild-type BRCA1/2 gene mutational status and an immune signature below a threshold level.

5. The method of claim 1, wherein the BRCA1/2 gene mutational status is selected from wild-type and mutated and the method further comprises designating the subject as a good responder or a poor responder to a therapy with a platinum-based agent based on the BRCA1/2 gene mutational status of the tumor sample.

6. The method of claim 5, wherein:
the subject is designated as a good responder if the tumor sample has a mutated BRCA1/2 gene mutational status;
the subject is designated as a good responder if the tumor sample has a wild-type BRCA1/2 gene mutational status and the immune signature is above the threshold level; or
the subject is designated as a poor responder if the tumor sample has a wild-type BRCA1/2 gene mutational status and the immune signature is below the threshold level.

7.-8. (canceled)

9. The method of claim 5 further comprising selecting the subject for a therapy with a platinum-based agent if the subject is a good responder.

10. The method of claim 1, wherein the immune signature is a M1 macrophage immune signature generated using an algorithm capable of estimating abundances of member cell types in a mixed cell population using gene expression data.

11. The method of claim 1, wherein the immune signature is an immune cell-specific enrichment score generated using an algorithm capable of calculating separate enrichment scores for each pairing of a sample and gene set, wherein each enrichment score represents the degree to which genes in a particular gene set are coordinately up-regulated or down-regulated within a sample.

12. (canceled)

13. The method of claim 1, wherein the immune signature is based on the measurement of one or more immune gene set.

14. The method of claim 1, wherein the tumor sample is from a subject with breast cancer.

15. The method of claim 1, wherein the tumor sample is from a subject with ovarian carcinoma (OV).

16. The method of claim 2, wherein the platinum-based agent is selected from the group consisting of oxaliplatin, cisplatin, carboplatin, nedaplatin, picoplatin, phenanthriplatin, triplatin, spiroplatin, satraplatin, iproplatin, and satraplatin.

17. The method of claim 2, wherein the therapy is a combination therapy that comprises at least one agent in addition to the platinum-based agent.

18. The method of 17, wherein the combination therapy comprises a taxane.

19. The method of 18, wherein the taxane is selected from the group consisting of paclitaxel, docetaxel, and cabazitaxel.

20. The method of claim 17, wherein the combination therapy comprises cisplatin or carboplatin.

21. (canceled)

22. A method comprising:
assaying a primary tumor sample for promoter methylation status of a wild-type BRCA1 gene from a subject prior to the subject receiving a therapy; and
(a) selecting the subject for a therapy with a platinum-based agent if the tumor sample has a promoter methylation status of fully methylated, or
(b) selecting the subject for an alternative therapy without a platinum-based agent or no therapy if the tumor sample has a promoter methylation status of partially methylated or unmethylated.

23. A method comprising:
assaying a recurrent tumor sample for promoter methylation status of a wild-type BRCA1 gene from a subject, wherein a primary tumor sample from the subject was previously assayed for promoter methylation status of a wild-type BRCA1 gene, and wherein the promoter methylation status of the primary tumor was fully methylated; and
(a) selecting the subject for a therapy with a platinum-based agent if the recurrent tumor sample has a promoter methylation status of fully methylated, or
(b) selecting the subject for an alternative therapy without a platinum-based agent if the recurrent tumor sample has a promoter methylation status of partially methylated or unmethylated.

24.-33. (canceled)

34. The method of claim 14, wherein the subject has triple negative breast cancer (TNBC).
